(19) United States
(12) Patent Application Publication
(10) Pub. No.: US 2022/0348960 A1
(43) Pub. Date: Nov. 3, 2022

(54) VIRAL MEDIATED GENE DELIVERY FOR TRANSIENT EXPRESSION OF PROTEINS

(71) Applicant: Regents of the University of Minnesota, Minneapolis, MN (US)

(72) Inventors: Christopher Stach, Minneapolis, MN (US); Michael Smanski, Minneapolis, MN (US); Ryan Langlois, Minneapolis, MN (US)

(21) Appl. No.: 17/726,116

(22) Filed: Apr. 21, 2022

Related U.S. Application Data
(60) Provisional application No. 63/177,761, filed on Apr. 21, 2021.

(51) Int. Cl. C12N 15/86; A61P 31/04; A61K 38/48

(52) U.S. Cl. CPC C12N 15/86; A61P 31/04; A61K 38/4886; C12Y 304/24075; C12N 2760/16122; C12N 2760/16132; C12N 2760/16142; C12N 2760/16162

(57) ABSTRACT

In certain embodiments, the present invention provides isolated replication defective non-integrating segmented viruses comprising a genome where at least one of the viral segments comprises a heterologous nucleotide sequence comprising a nucleotide segment that encodes an effector protein. Also provided are methods of using these viruses.

### CROSS-REFERENCE TO RELATED APPLICATION

This application claims priority to U.S. Provisional Application No. 63/177,761 that was filed on Apr. 21, 2021. The entire content of this application referenced above is hereby incorporated by reference herein.

### SEQUENCE LISTING

The instant application contains a Sequence Listing which has been submitted electronically in ASCII format and is hereby incorporated by reference in its entirety. Said ASCII copy, created on Jul. 6, 2022, is named 09531_537US1_SL.txt and is 66,068 bytes in size.

### BACKGROUND

Staphylococcus aureus is gram-positive coccus that colonizes the skin and mucosal surfaces of up to 40% of the human population at any given time. In the U.S. yearly, S. aureus accounts for approximately 5,000 cases of toxic shock syndrome (TSS), 70,000 cases of pneumonia, 40,000 cases of infective endocarditis (IE), and more than 500,000 post-surgical infections. S. aureus is the leading cause of IE and the second leading cause of sepsis. S. aureus has acquired resistance to methicillin (MRSA) and vancomycin (VISA and VRSA) making it very difficult to treat. To combat these and other antibiotic S. aureus strains new antimicrobials and delivery methods must be developed.

Antibiotic treatment requires delivery of the agent to the site of infection in a concentration high enough to eliminate bacteria at the site of infection altogether or slow bacterial growth so that a secondary agent can be used to eliminate the bacteria. Currently, it is difficult to deliver specific antibiotic agents due to their instability in serum, susceptibility to enzymes, e.g., proteases, and a general requirement for high localized concentrations.

### SUMMARY

As disclosed herein, influenza A virus (IAV), a negative sense RNA virus which primarily replicates in epithelial cells of the upper and lower respiratory tract but can also infect a spectrum of other cell lineages, was employed to deliver effector proteins, such as a protein antibiotic. Because infected cells are eliminated through two major mechanisms, apoptosis/necrosis driven by virus replication or clearance mediated through the innate and adaptive arms of the immune system, the transient nature of infection and absence of possible genome integration are advantages to IAV as a method of gene delivery.

Thus, the disclosure provides for the use of a non-integrating virus, e.g., influenza virus, to infect, and express transgenes which encode effector proteins, such as antibiotics, in eukaryotic cells. The protein encoded by the transgene may contain a secretion signal which allows for secretion of the effector protein out of the cell. Because influenza virus is an RNA virus the transgene will not integrate, thus inhibiting or preventing prolonged expression. In one embodiment, the virus may be replication deficient, which results in a single cycle of infection. In one embodiment, the tropism of the virus may be modified by co-expressing heterologous ligands (e.g., receptors), to target tissues of interest more precisely. For example, in influenza virus, the heterologous ligand may replace one or more of the influenza virus glycoproteins, HA and NA.

Viruses useful in the transient delivery of an effector protein, such as an antibiotic protein, to cells include but are not limited to influenza A virus, influenza B virus, influenza C virus, influenza D virus, isavirus, quaranjavirus or thogotovirus, as well as viruses that infect bacteria (phage), e.g., positive-sense single-stranded RNA phage such as MS2 phage, bacteriophage f2, bacteriophage Qβ or any of the Leviviridae family of phages.

In one embodiment, a non-integrating virus, e.g., influenza virus, may be modified to include sequence to express two different effector (e.g., two different antibiotic) proteins. For example, two different viral segments in a segmented virus may be modified, each with sequences for a different effector (e.g., antibiotic) protein. Alternatively, one viral segment in a segmented virus is modified with sequences for two different effector (e.g., two different antibiotic) proteins. In one embodiment, the virus would express an endolysin and a holin, or lysostaphin and a defensin. These viruses may be used to treat polymicrobial infections and also as a means to overcome possible resistance.

Therefore, a method for preventing, inhibiting, or treating a bacterial infection in an animal is provided. In one embodiment, the animal is an avian. In one embodiment, the animal is a mammal, e.g., a human or other primate, swine, caprine, ovine, canine, feline, equine or bovine. In one embodiment, the infection is caused by antibiotic resistant microbes, e.g., resistant to a non-protein antibiotic. In one embodiment, the method includes administering to the animal an effective amount of a composition comprising a recombinant virus, e.g., a replication defective influenza virus or recombinant phage (the latter of which can directly infect bacterial cells in a an avian or mammalian host). In one embodiment, the composition is systemically administered. In one embodiment, the composition is locally administered. In one embodiment, the composition is administered to the respiratory system, e.g., via inhalation. In one embodiment, the composition is topically delivered, e.g., in a bandage having the composition. In one embodiment, the mammal has a bacterial infection. In one embodiment, the mammal has a Staphylococcus, Proteus, Listeria, or Pseudomonas infection. In one embodiment, the mammal has a fungal infection. In one embodiment, the antimicrobial protein comprises a lysostaphin cutinase-like serine esterase, defensin, indolicidin, protegrin, LL-37, S-type pyocin or tailocin (e.g., endolysin or holin). In one embodiment, the antimicrobial protein comprises one of SEQ ID Nos. 1-10 or 29-37, or a protein or peptide with at least 80% amino acid sequence identity to a portion there of with antimicrobial activity. In one embodiment, the antimicrobial protein comprises PlyF307, PlyG, PlyPH, artilysin, ClyS, lambdaSa2-E-lyso-SH3b, LysK/CHAPk, LysGH15, MV-L, PhiSH2, Phi11, PlySs2,'Ply187AN-KSH3b, SAL-1, Twort, WMY, 80alpha, 2638A, PlyGBS,/PlyGBS90-1, PlySK1249, Cpl-1, Cpl-771, PAL, PlyC, PlyPy, P128, endoE, P22sTsp (see Roach and Donovan, Bacteriophage, 5:3 (2015), Table 1, which is incorporated by reference herein).

### BRIEF DESCRIPTION OF FIGURES

The patent or application file contains at least one drawing executed in color. Copies of this patent or patent application publication with color drawings will be provided by the Office upon request and payment of the necessary fee.

FIG. 1. Amino acid sequence of gluc_lyso (SEQ ID NO:1) FIG. 2. pCSS_gluc_IAVlyso plasmid. The lysostaphin gene, represented as the upper block arrow, is expressed in the antisense orientation by a human polymerase-I promoter, represented by the thin black arrow. Murine polymerase-I terminator is depicted as inverted T.

FIG. 3. Functional assays of lysostaphin generated through IAV RDRP. A mixture of 5 plasmids comprising pDZ PR8 PA, PB1, PB2, NP and pCSS_gluc_IAVlyso were transfected into HEK293T cells. A GFP encoding plasmid was transfected as a negative control. The supernatant was harvested after 48 hrs. To assay lysostaphin activity, 30 ┌L was spotted onto a plate overlayed with methicillin resistant S. aureus (MRSA) and clearance was determined after 24 hours. RDRP+lysostaphin=pDZ PR8 PA, PB1, PB2, NP and pCSS_glue_IAVlyso; RDRP only=pDZ PR8 PA, PB1, PB2, NP; GFP+RDRP=pDZ PR8 PA, PB1, PB2, NP and GFP; IAV lysostaphin=pCSS_gluc_IAVlyso; Cells only=supernatant from non transfected cells; Lipofectamine=lipofectamine only treated cell supernatant.

FIG. 4. Rescue of single cycle gluc_IAVlyso virus. Lysostaphin encoding virus was rescued using pDZ plasmids encoding PA, PB1, PB2, NP, NS, M, NA with the addition of pCSS_glue_IAVlyso and pCAGGs WSN HA.

FIG. 5. gluc_IAVlyso recombinant virus infection leads to secretion of functional lysostaphin. MDCK or HEK293T cells were infected with the recombinant gluc_IAVlyso virus to determine infectivity and ability to produce lysostaphin. The supernatant was harvested after 48 hrs. To assay lysostaphin activity, 30 ┌t, was spotted onto a plate overlayed with methicillin resistant S. aureus (MRSA) and clearance was determined after 24 hours. Labels next to plate (HEK293T, MDCK) indicate origin of supernatant.

FIG. 6. In vivo IAV lysostaphin expression. Mice were sedated with ketamine/xylazine and infected intranasally with 50 μl of 10⁵ PFU of gluc_IAVlyso (IAV Lyso) or GFP (IAV GFP) influenza recombinant virus. Lung homogenate ELISA indicated therapeutic levels of lysostaphin produced after gluc_IAVlyso infection. IAV Lyso=32.6 ng/mL (+/−3.4 ng/mL SD); IAV GFP=1.3 ng/mL (+/−0.035 ng/mL SD).

FIG. 7. Murine model of pneumonia treated with Lysostaphin expressing recombinant IAV. MRSA pneumonia was treated with IAV lysostaphin recombinant virus to identify decrease in bacterial titer due to expression of lysostaphin.

FIG. 8. Single cycle IAV assay for the expression of secreted lysostaphin in HEK293T and MDCK cells. Viral infection of HEK293T cells and MDCK cells. Virus infection leads to production of lysostaphin in concentrations high enough to lyse Staphylococcus aureus. Supernatant from infected cells was spotted onto plates overlayed with Staphylococcus aureus. Top left: supernatant from no infection control; top right supernatant from cells infected with IAV encoding lysostaphin gene; bottom left: supernatant from cells infected with IAV encoding GFP; bottom right supernatant from negative viral packaging control.

FIG. 9. In vivo lysostaphin expression.

FIGS. 10A-10D. Key components of IAV and its life cycle. FIG. 10A. Illustration of the 8 segment IAV, genomic organization and associated proteins. FIG. 10B. A schematic of RNA species generated through the course of a cellular infection by IAV. vRNA is transcribed into cRNA which then is used to generate more vRNA for replication. The vRNA is also transcribed into mRNA for protein translation. FIG. 10C. A simplified schematic of the IAV life cycle upon infecting a host cell. FIG. 10C. Viral genetic material is transported into the nucleus where it is replicated and used to template mRNA for protein production. Protein bound vRNA is packaged into newly budding viral particles to continue the life cycle. FIG. 10D. rIAV loses its ability to replicate without human intervention, prohibiting pathogenesis. rIAV can then be used to produce effector proteins for therapeutic, research, or manufacturing applications.

FIG. 11A-11C. FIG. 11A. Strand specific qRT-PCR analysis to quantify RNA species in rIAV infected cells. Bar graphs show data from rIAV infected HEK293t cells. Heatmaps serve as projections of bar graphs data for two additional cell lines (A549 and MDCK) as well as a no-cell control. Top: GFP targeting primers confirm lifecycle progression in cells infected with GFP encoding virus. Bottom: Lysostaphin targeting primers confirm lifecycle progression in cells infected with Lysostaphin encoding virus. FIG. 11B Single cells were collected from a population infected with GFP encoding rIAV at low dosage (0.1 MOI). Survival was recorded for GFP positive (infected), GFP negative (uninfected), and untreated cells. FIG. 11C. rIAV was stored at −80° C., −20° C., 4° C., and 22° C. for varied times. 10⁶ cells were infected at each time point to measure viral decay.

FIGS. 12A-12D. rIAV platform produces functional antimicrobial proteins in vitro and in vivo. FIG. 12A. Supernatant from lysostaphin encoding rIAV inhibits MRSA MW2 growth on a comparable to 1 μg of Kanamycin, while dilutions of control treatments were unable to generate similar inhibition. FIG. 12B. Cell culture treated with lysostaphin encoding rIAV at various dosages was contaminated with MRSA MW2 and OD600 was measured 24 hr later as a proxy for bacterial growth. Lysostaphin encoding rIAV was able to completely inhibit the growth of MRSA MW2. FIG. 12C. Mice treated intranasally with lysostaphin encoding rIAV produced lysostaphin in their lungs at inhibitory concentrations. FIG. 12D. Liquid Chromatography data on cell media from cells infected with FK-13 antimicrobial peptide encoding rIAV. A double peak at ˜3.15 retention time is present for FK-13 rIAV treated media but not GFP rIAV or untreated cells indicating production of FK-13.

FIG. 13. S. aureus inhibition in cell culture. Left column, treatment with virus alone does not impact OD600 readings. Right column top, contaminating cells treated with GFP virus leads to increase of OD600 readings. Right column bottom, at >0.005 MOI of iAS008 growth of S. aureus is fully suppressed.

FIG. 14. This is the same data as in FIG. 13, but with hill function fit. Hill coefficient: 0.48. EC50: 0.0024 MOI.

FIG. 15. Mice treated intranasally with lysostaphin encoding rIAV produced lysostaphin in their lungs at inhibitory concentrations.

FIGS. 16A-16B. FIG. 16A. Left panel: negative strand GFP qRT-PCR; Right panel: positive strand GFP qRT-PCR. FIG. 16B. Left panel: negative strand Lysostaphin qRT-PCR; Right panel: positive strand Lysostaphin qRT-PCR.

FIG. 17. The rIAV platform serves as an alternative pipeline for traditional biomanufacturing. Top: Traditionally, cell lines must be transformed, selected for production, and grown to scale before protein is collected. Long (˜24 hr) doubling times are exasperated if the cell line has any fitness penalty that comes with protein production. Bottom: Wild-type cell lines can be pre-expanded at normal doubling times ahead of production. Addition of rIAV rapidly reprograms cells for biomanufacturing without expansion delays.

### DETAILED DESCRIPTION

### Definitions

As used herein, the term “isolated” refers to in vitro preparation and/or isolation of a nucleic acid molecule, e.g., vector or plasmid, peptide or polypeptide (protein), or virus, so that it is not associated with in vivo substances, or is substantially purified from in vitro substances. An isolated virus preparation is generally obtained by in vitro culture and propagation, and/or via passage in eggs for influenza virus and is substantially free from other infectious agents.

As used herein, “substantially purified” means the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome. Reassortant viruses, e.g., reassortant influenza viruses, can be prepared by recombinant or nonrecombinant techniques.

As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

As used herein, a “heterologous” influenza virus gene or gene segment is from an influenza virus source that is different than a majority of the other influenza viral genes or gene segments in a recombinant, e.g., reassortant, influenza virus.

The terms “isolated polypeptide,” “isolated peptide” or “isolated protein” include a polypeptide, peptide or protein encoded by cDNA or recombinant RNA including one of synthetic origin, or some combination thereof.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Alignments using these programs can be performed using the default parameters. Software for performing BLAST analyses is publicly available through the National Center for Biotechnology Information (http://www.ncbi.nlm.nih.gov/). The algorithm may involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm may also perform a statistical analysis of the similarity between two sequences. One measure of similarity provided by the BLAST algorithm may be the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The BLASTN program (for nucleotide sequences) may use as defaults a wordlength (W) of 11, an expectation (E) of 10, a cutoff of 100, M=5, N=−4, and a comparison of both strands. For amino acid sequences, the BLASTP program may use as defaults a wordlength (W) of 3, an expectation (E) of 10, and the BLOSUM62 scoring matrix. See http://www.ncbi.nlm.nih.gov. Alignment may also be performed manually by inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### Abbreviations

rIAV: recombinant Influenza A Virus

sciIAV: single cycle infectious Influenza A Virus

HA: hemagglutinin

MDCK-HA: Madin-Darby Canine Kidney Cell expressing IAV hemagglutinin

RBD: Receptor binding domain

GFP: Green Fluorescent Protein

MRSA: Methicillin-resistant Staphylococcus aureus

RT-PCR: reverse transcriptase-polymerase chain reaction

qRT-PCR: quantitative reverse transcriptase-polymerase chain reaction

### Influenza Virus Structure and Propagation

Influenza A viruses, for example, possess a genome of eight single-stranded negative-sense viral RNAs (vRNAs) that encode at least ten proteins. The influenza virus life cycle begins with binding of the hemagglutinin (HA) to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nucleoprotein (NP), which encapsidates vRNA, and the viral polymerase complex, which is formed by the PA, PB1, and PB2 proteins. RNPs are transported into the nucleus, where transcription and replication take place. The RNA polymerase complex catalyzes three different reactions: synthesis of an mRNA with a 5′ cap and 3′ polyA structure, of a full-length complementary RNA (cRNA), and of genomic vRNA using the cRNA as a template. Newly synthesized vRNAs, NP, and polymerase proteins are then assembled into RNPs, exported from the nucleus, and transported to the plasma membrane, where budding of progeny virus particles occurs. The neuraminidase (NA) protein plays a crucial role late in infection by removing sialic acid from sialyloligosaccharides, thus releasing newly assembled virions from the cell surface and preventing the self-aggregation of virus particles. Although virus assembly involves protein-protein and protein-vRNA interactions, the nature of these interactions is largely unknown.

Although influenza B and C viruses are structurally and functionally similar to influenza A virus, there are some differences. For example, influenza C virus has only seven gene segments.

### Exemplary Methods and Compositions

Antibiotic treatment requires delivery of the agent to the site of infection in a concentration high enough to eliminate bacteria at the site of infection altogether or slow bacterial growth so that a secondary agent can be used to eliminate the bacteria. Currently, it is difficult to deliver specific antibiotic agents such as protein antibiotics due to their instability in serum, susceptability to proteases, and general requirement for high localized concentrations. The present system addresses these issues by co-opting the native machinery of the mammalian cell to produce the antibiotic locally and transiently. The concentrations locally should be high enough to kill or slow the growth of the bacteria and the protein antibiotics are essentially targeted to the area of bacterial infection to increase specificity of treatment. The system allows for the localized delivery of protein antibiotics some of which are not used due to drawbacks in delivery and effective concentration. The system opens the door to using new classes of antibiotics and overcomes current drawbacks which are limiting the use of some antibiotics.

Currently, the use of protein antibiotics has been done by systemic, intravenous, as well as localized topical treatments. These methods have critical limitations, (i) the proteins must be produced, purified, and retain activity after purification, (ii) the protein must be stable enough to be injected or supplemented into a topical treatment and the effective concentration should be at a level such that it kills the bacteria, and (iii) the treatments are more generalized and systemic. The method described herein produces the protein from the mammalian cells of the infected tissues bypassing purification, stability, and concentration issues. The system is also specific in that you can target the area of infection and deliver the treatment locally which allows for high localized concentrations of the antibiotic protein.

In one embodiment, the method treats infections at the site of bacterial colonization. The use of the eukaryotic cells to transiently express protein based antibiotics locally provides for local delivery of the therapeutic. The influenza virus expresses the transgene but does not replicate or spread as in a native infection. In one embodiment, the transgene encodes a protein antibiotic. In one embodiment, the transgene encodes lysostaphin which specifically lyses Staphylococcus aureus. In one embodiment, the transgene encodes a protein that is a can bacterial cell specific lytic agent. In one embodiment, the transgene encodes an antimicrobial peptide. In one embodiment, the transgene encodes a protein with a modified sequence to allow for efficient expression and secretion out of cells, e.g., mammalian cells. The combined system of transgene selection and construction, IAV mediated expression, and transient targeted expression together constitute an antibiotic delivery method.

As described herein, lysostaphin, which is specific to Staphylococcus aureus, can be secreted from mammalian cells using a genetic construct, the viral replication machinery and mammalian cells. The secreted lysostaphin is active and inhibits growth as well as lyses S. aureus. In one embodiment, the IAV that delivers the gene encoding a protein antibiotic is a non replicating viral vector that infects mammalian cells (e.g., HEK 293T and MDCK) that expresses the protein which was shown to be active through functional S. aureus lytic assays. The protein was also expressed in vivo and its concentration quantified in a mouse model. This data demonstrate that the system functions successfully in vivo and produces functional lysostaphin.

The rIAV can deliver proteins such as SsoPox, an organophosphate degrading enzyme, that can be used prophylactically or as a post-exposure therapy to degrade and modify chemical in the body including toxins (such as paraoxon, VX, and sarin) or bacterial signaling molecules such as N-(3-oxododecanoyl)-homoserine lactone.

The rIAV can be designed to express proteins that inhibit heterologous viral entry to cells through competitive binding to cellular receptors (e.g., SARS-CoV-2 S RBD), binding of heterologous viral glycoproteins or membrane (e.g., sACE2, Anti-SARS-CoV-2 ScFv), through stimulation of patient immune response (e.g., MBL), or through generation of protease inhibitors. This could serve as a prophylactic treatment or as a post-exposure therapy to limit viral replication.

The rIAV can be designed for in vitro biomanufacturing protein products of economic interest (e.g., erythropoeitin for treatment of anemia, SARS-CoV-2 S RBD for development of antigen testing).

The rIAV can be used to produce medically relevant proteins in the body for treatment of transient or chronic afflictions (e.g., MBL for MBL deficiency, erythropoietin for drug induced anemia).

### Exemplary Effector Proteins

Exemplary Anti-Microbial Proteins

In one embodiment, the anti-microbial protein expressed by a recombinant virus or phage is a cutinase-like serine esterase, e.g., a trehalose dimycolate hydrolase (TDMH) for the treatment of Mycobacteria infections e.g., M. tuberculosis, Mycobacterium bovis, Mycobacterium marinum, M. smegmatis or Mycobacterium avium infections. In one embodiment, the anti-microbial protein is an antimicrobial peptide (AMP), for example, a broad-spectrum antibacterial peptide such as a defensin, indolicidin, protegrin or LL-37, an antifungal protein, e.g., protegrin, indolicidin or histatin, an anti-viral protein (e.g., an anti-HIV, HSV, or VSV protein) such as indolicidin, protegrin or defensin, a S-type pyocin, e.g., to prevent, inhibit or treat Gram-negative bacterial infections such as P. aeruginosa infection, or a tailocin, e.g., to prevent, inhibit or treat Gram positive and negative bacterial infection, e.g., infection by Listeria, Proteus vulgaris, or P. aeruginosa.

In one embodiment, the antimicrobial protein comprises:

(SEQ ID NO: 2)
AAT HEHSAQWLNN YKKGYGYGPY PLGINGGMHY
GVDFFMNIGT PVKAISSGKI VEAGWSNYGG GNQIGLIEND
GVHRQWYMHL SKYNVKVGDY VKAGQIIGWS GSTGYSTAPH
LHFQRMVNSF SNSTAQDPMP FLKSAGYGKA GGTVTPTPNT
GWKTNKYGTL YKSESASFTP NTDIITRTTG PFRSMPQSGV
LKAGQTIHYD EVMKQDGHVW VGYTGNSGQR IYLPVRTWNK
STNTLGVLWG TIK

or a sequence having at least 80%, 85%, 90%, 92%, 94%, 95%, 96%, 97%, 98% or 99% identity thereto.

In one embodiment, the antimicrobial protein comprises:

(SEQ ID NO: 3)
VETSKAPVENT AEVETSKALV QNRTALRAAT HEHSAQWLNN
YKKGYGYGPY PLGINGGMHY GVDFFMNIGT PVKAISSGKI
VEAGWSNYGG GNQIGLIEND GVHRQWYMHL SKYNVKVGDY
VKAGQIIGWS GSTGYSTAPH LHFQRMVNSF SNSTAQDPMP
FLKSAGYGKA GGTVTPTPNT GWKTNKYGTL YKSESASFTP
NTDIITRTTG PFRSMPQSGV LKAGQTIHYD EVMKQDGHVW
VGYTGNSGQR IYLPVRTWNK STNTLGVLWG TIK

or a sequence having at least 80%, 85%, 90%, 92%, 94%, 95%, 96%, 97%, 98% or 99% identity thereto.

In one embodiment, the antimicrobial protein comprises:

(SEQ ID NO: 4)
MVLQQTEPTD GADRKASDGP LTVTAPVPYA AGPTLRNPFP
PIADYGFLSD CETTCLISSA GSVEWLCVPR PDSPSVFGAI
LDRGAGHFRL GPYGVSVPAA RRYLPGSLIL ETTWQTHTGW
LIVRDALVMG PWHDIDTRSR THRRTPMDWD AEHILLRTVR
CVSGTVELVM SCEPAFDYHR VSATWEYSGP AYGEAIARAS
RNPDSHPTLR LTTNLRIGIE GREARARTRL TEGDNVFVAL
SWSKHPAPQT YEEAADKMWK TSEAWRQWIN VGDFPDHPWR
AYLQRSALTL KGLTYSPTGA LLAAPTTSLP ETPQGERNWD
YRYSWIRDST FALWGLYTLG LDREADDFFS FIADVSGANN
GERHPLQVMY GVGGERSLVE EELHHLSGYD NSRPVRIGNG
AYNQRQHDIW GTMLDSVYLH AKSREQIPDA LWPVLKNQVE
EAIKHWKEPD RGIWEVRGEP QHFTSSKIMC WVALDRGSKL
AELQGEKSYA QQWRAIAEEI KADVLARGVD KRGVLTQRYG
DDALDASLLL AVLTRFLPAD DPRIRATVLA IADELTEDGL
VLRYRVEETD DGLAGEEGTF TICSFWLVSA LVEIGEISRA
KHLCERLLSF ASPLHLYAEE IEPRTGRHLG NFPQAFTHLA
LINAVVHVIR AEEEADSSGV FVPANAPM

or a sequence having at least 80%, 85%, 90%, 92%, 94%, 95%, 96%, 97%, 98% or 99% identity thereto.

In one embodiment, the antimicrobial protein comprises:

(SEQ ID NO: 5)
MKFFVLVAIA FALLACVAQA QPVSDVDPIP EDHVLVHEDA
HQEVLQHSRQ KRATCDLLSK WNWNHTACAG HCIAKGFKGG
YCNDKAVCVC RN

or a sequence having at least 80%, 85%, 90%, 92%, 94%, 95%, 96%, 97%, 98% or 99% identity thereto.

In one embodiment, the antimicrobial protein comprises:

(SEQ ID NO: 6)
ILPWKWPWWP WRR

or a sequence having at least 80%, 85%, 90%, 92%, 94%, 95%, 96%, 97%, 98% or 99% identity thereto.

In one embodiment, the antimicrobial protein comprises:

(SEQ ID NO: 7)
MKFFVFALVL ALMISMISAD SHEKRHHGYR RKFHEKHHSH
REFPFYGDYG SNYLYDN

or a sequence having at least 80%, 85%, 90%, 92%, 94%, 95%, 96%, 97%, 98% or 99% identity thereto.

In one embodiment, the antimicrobial protein comprises:

(SEQ ID NO: 8)
METQRASLCL GRWSLWLLLL ALVVPSASAQ ALSYREAVLR
AVDRLNEQSS EANLYRLLEL DQPPKADEDP GTPKPVSFTV
KETVCPRPTR QPPELCDFKE NGRVKQCVGT VTLDQIKDPL
DITCNEVQGV RGGRLCYCRR RFCVCVGRG

or a sequence having at least 80%, 85%, 90%, 92%, 94%, 95%, 96%, 97%, 98% or 99% identity thereto.

In one embodiment, the antimicrobial protein comprises:

(SEQ ID NO: 9)
MAQQTHYDGT SGSVVITSGP PASSGGSGGG FGGGGGVSGG
FGRTSKRRQK ARRRAIEADR QKKEKERAQA EAEAQAEAAQ
AQAAAAQAQE QARVQARHQQ LEGLAQHHAA VRVEVDQRFA
ARSAQLAPTL EQEVLAARRP PDIHLSERLQ LHNITKQKQE
VDGLITRKTA ELNAKNAVAR SFDGHDPLTR TVNDYRARLE
QFGEALVHGH QTWENAYNAA HEARLLSTQI SALTGKSSAL
ARRHAEQTIV WREREAVWEA QRQYARQREA RVRFKQQADE
DARVERVRQA NTLTVPVSSL TAGGMALTRD GIFVAQQGAA
VLEMAVQSAV NTLIDFGRIA AKTGPVFVTA MVYSPTLGDG
ELSAEQRRRL FQAVGVPAPT LGLTDRQALQ SVADAGGSVE
VAYRLKSETV PEGTAIIAVA TGDAIGADVP VINAVLDPLT
GLYSAEIPGS PARHLQFTPD TTQVATASQP GLSVLTPQVE
AIAAGVDLRI SDCIVCVPDQ PPIYFTFNLP PIGSGVVTGV
GKPAANDWWK AASQAQGAAI PVQIGDQFRG REFNSLAAFD
EALWRTLGEH PTLIAQFDEV NKKRIEQGFA PYAPKSTWVG
ERREFELRYQ ERAELGANPF NLDKISITTP QSIQGRLGIT
PAVLPWPIRP VGVGTWTPLV PPGIEHLGPT TLPVTPSIPA
VYPGTPIIPV LPQNETFPAV DEGQIGASIP GYPADMELPS
PDVLFLDRRD DPGVAMGVGQ SVSGIWLGDA ARTEGAPIPE
QIADKLRSME FRNFHGFRRA FWRAVAADAG LSQQFSKSNL
DRMRDGSAPF SVLVDQVGGR KAFELHHQVE ISKGGDVYGL
DNISVMTPKR HINLHKENNQ

or a sequence having at least 80%, 85%, 90%, 92%, 94%, 95%, 96%, 97%, 98% or 99% identity thereto.

In one embodiment, the antimicrobial protein comprises:

(SEQ ID NO: 10)
MENKESGEKF SWLEVFKQVV ISGFTGFLAG IYGYEQGYSE
FITMAFSGLG GALGGHLLDL LWKRLTNSLE KENHSKH

or a sequence having at least 80%, 85%, 90%, 92%, 94%, 95%, 96%, 97%, 98% or 99% identity thereto.

In one embodiment, the antimicrobial protein comprises:

(SEQ ID NO: 29)
EPCSDIEVVFARGTSEPAGIGRVGQALTDAIRNQVGGRTVSTYGVNYPAT
YDFLAAADGANDATNRIATLAEQCPSTRVVLGGYSQGAAVVDMLLGIPPL
GNKVGNFGSAPPLPSNLMNNVAAVAVFGNPSAKFGIPVTSRFGGRAIDAC
SDGDPICSDGRNPFAHTHYESSPFIPQAAGLIAGLV

or a sequence having at least 80%, 85%, 90%, 92%, 94%, 95%, 96%, 97%, 98% or 99% identity thereto.

In one embodiment, the antimicrobial protein comprises:

(SEQ ID NO: 30)
MKTQRDGHSLGRWSLVLLLLGLVMPLAIIAQVLSYKEAVLRAIDGINQRS
SDANLYRLLDLDPRPTMDGDPDTPKPVSFTVKETVCPRTTQQSPEDCDFK
KDGLVKRCMGTVTLNQARGSFDISCDKDNKRFALLGDFFRKSKEKIGKEF
KRIVQRIKDFLRNLVPRTES

or a sequence having at least 80%, 85%, 90%, 92%, 94%, 95%, 96%, 97%, 98% or 99% identity thereto.

In one embodiment, the antimicrobial protein comprises:

(SEQ ID NO: 31)
RGGRLCYCRRRFCVCVGRX

or a sequence having at least 80%, 85%, 90%, 92%, 94%, 95%, 96%, 97%, 98% or 99% identity thereto.

In one embodiment, the antimicrobial protein comprises:

(SEQ ID NO: 32)
ILPWKWPWWPWRR

or a sequence having at least 80%, 85%, 90%, 92%, 94%, 95%, 96%, 97%, 98% or 99% identity thereto.

In one embodiment, the antimicrobial protein comprises:

(SEQ ID NO: 33)
MKFFVFALVLALMISMISADSHEKRHHGYRRKFHEKHHSHREFPFYGDYG
SNYLYDN

or a sequence having at least 80%, 85%, 90%, 92%, 94%, 95%, 96%, 97%, 98% or 99% identity thereto.

In one embodiment, the antimicrobial protein comprises:

(SEQ ID NO: 34)
MRTLAILAAILLVALQAQAEPLQARADEVAAAPEQIAADIPEVVVSLAWD
ESLAPKHPGSRKNMACYCRIPACIAGERRYGTCIYQGRLWAFCC

or a sequence having at least 80%, 85%, 90%, 92%, 94%, 95%, 96%, 97%, 98% or 99% identity thereto.

In one embodiment, the antimicrobial protein comprises:

(SEQ ID NO: 35)
MRIHYLLFALLFLFLVPVPGHGGIINTLQKYYCRVRGGRCAVLSCLPKEE
QIGKCSTRGRKCCRRKK

or a sequence having at least 80%, 85%, 90%, 92%, 94%, 95%, 96%, 97%, 98% or 99% identity thereto.

In one embodiment, the antimicrobial protein comprises:

(SEQ ID NO: 36)
MSNDNEVPGS MVIVAQGPDD QYAYEVPPID SAAVAGNMFG
DLIQREIYIQ KNIYYPVRSI FEQGTKEKKE INKKVSDQVD
GLLKQITQGK REATRQERVD VMSAVLHKME SDLEGYKKTE
TKGPFIDYEK QSSLSIYEAW VRIWEKNSWE ERKKYPFQQL
VRDELERAVA YYKQDSLSEA VKVLRQELNK QKALKEKELL
SQLERDYRTR KANLEMKVQS ELDQAGSALP PLVSPTPEQW
LERATRLVTQ AIADKKQLQT TNNTLIKNSP TPLEKQKAIY
NGELLVDEIA SLQARLVKIN AETTRRRTEA ERKAAEEQAL
QDAIKFTADF YKEVTEKFGA RTSEMARQLA EGARGKNIRS
SAEAIKSFEK HKDALNKKLS LKDRQAIAKA FDSLDKQMMA
KSLEKFSKGF GVVGKAIDAA SLYQEFKIST ETGDWKPFFV
KIETLAAGAA ASWLVGIAFA TATATPIGIL GFALVMAVTG
AMIDEDLLEK ANNLVISI

or a sequence having at least 80%, 85%, 90%, 92%, 94%, 95%, 96%, 97%, 98% or 99% identity thereto.

In one embodiment, the antimicrobial protein comprises:

(SEQ ID NO: 37)
MEIQKKLVDPSKYGTKCPYTMKPKYITVHNTYNDAPAENEVSYMISNNN
EVSFHIAVDDKKAIQGIPLERNAWACGDGNGSGNRQSISVEICYSKSGG
DRYYKAEDNAVDVVRQLMSMYNIPIENVRTHQSWSGKYCPHRMLAEGRW
GAFIQKVKNGNVATTSPTKQNIIQSGAFSPYETPDVMGALTSLKMTADF
ILQSDGLTYFISKPTSDAQLKAMKEYLDRKGWWYEVK

or a sequence having at least 80%, 85%, 90%, 92%, 94%, 95%, 96%, 97%, 98% or 99% identity thereto.

For any of SEQ ID Nos. 1-10 or 29-37 if present, a native signal peptide may be removed or replaced with a heterologous signal peptide (see below for exemplary signal peptides).

Exemplary Effector Proteins

In one embodiment, the effector protein comprises:

(SEQ ID NO: 38)
MWWRLWWLLLLLLLLWPMVWANITNLCPFGEVFNATKFPSVYAWERKKI
SNCVADYSVLYNSTFFSTFKCYGVSATKLNDLCFSNVYADSFVVKGDDV
RQIAPGQTGVIADYNYKLPDDFMGCVLAWNTRNIDATSTGNYNYKYRYL
RHGKLRPFERDISNVPFSPDGKPCTPPALNCYWPLNDYGFYTTTGIGYQ
PYRVVVLSFELLNAPATV

or a sequence having at least 80%, 85%, 90%, 92%, 94%, 95%, 96%, 97%, 98% or 99% identity thereto.

In one embodiment, the effector protein comprises:

(SEQ ID NO: 39)
MSSSSWLLLSLVAVTAAQSTIEEQAKTFLDKFNHEAEDLFYQSSLASWN
YNTNITEENVQNMNNAGDKWSAFLKEQSTLAQMYPLQEIQNLTVKLQLQ
ALQQNGSSVLSEDKSKRLNTILNTMSTIYSTGKVCNPDNPQECLLLEPG
LNEIMANSLDYNERLWAWESWRSEVGKQLRPLYEEYVVLKNEMARANHY
LNEIMANSLDYNERLWAWESWREDVEHTFEEIKPLYEHLHAYVRAKLMN
AYPSYISPIGCLPAHLLGDMWGRFWTNLYSLTVPFGQKPNIDVTDAMVD
QAWDAQRIFKEAEKFFVSVGLPNMTQGFWENSMLTDPGNVQKAVCHPTA
WDLGKGDFRILMCTKVTMDDFLTAHHEMGHIQYDMAYAAQPFLLRNGAN
EGFHEAVGEIMSLSAATPKHLKSIGLLSPDFQEDNETEINFLLKQALTI
VGTLPFTYMLEKWRWMVFKGEIPKDQWMKKWWEMKREIVGVVEPVPHDE
TYCDPASLFHVSNDYSFIRYYTRTLYQFQFQEALCQAAKHEGPLHKCDI
SNSTEAGQKLFNMLRLGKSEPWTLALENVVGAKNMNVRPLLNYFEPLFT
WLKDQNKNSFVGWSTDWSPYADQSIKVRISLKSALGDKAYEWNDNEMYL
FRSSVAYAMRQYFLKVKNQMILFGEEDVRVANLKPRISFNFFVTAPKNV
SDIIPRTEVEKAIRMSRSRINDAFRLNDNSLEFLGIQPTLGPPNQPPVS

or a sequence having at least 80%, 85%, 90%, 92%, 94%, 95%, 96%, 97%, 98% or 99% identity thereto.

In one embodiment, the effector protein comprises:

(SEQ ID NO: 40)
MGVKVLFALICIAVAEADIQLTQSPDSLAVSLGERATINCKSSQSVLYS
SINKNYLAWYQQKPGQPPKLLIYWASTRESGVPDRFSGSGSGTDFTLTI
SSLQAEDVAVYYCQQYYSTPYTFGQGTKVEIKGGGGSGGGGSGGGGSQM
QLVQSGTEVKKPGESLKISCKGSGYGFITYWIGWVRQMPGKGLEWMGII
YPGDSETRYSPSFQGQVTISADKSINTAYLQWSSLKASDTAIYYCAGGS
GISTPMDVWGQGTTVTV

or a sequence having at least 80%, 85%, 90%, 92%, 94%, 95%, 96%, 97%, 98% or 99% identity thereto.

In one embodiment, the effector protein comprises:

(SEQ ID NO: 41)
MSLFPSLPLLLLSMVAASYSETVTCEDAQKTCPAVIACSSPGINGFPGK
DGRDGTKGEKGEPGQGLRGLQGPPGKLGPPGNPGPSGSPGPKGQKGDPG
KSPDGDSSLAASERKALQTEMARIKKWLTFSLGKQVGNKFFLTNGEIMT
FEKVKALCVKFQASVATPRNAAENGAIQNLIKEEAFLGITDEKTEGQFV
DLTGNRLTYTNWNEGEPNNAGSDEDCVLLLKNGQWNDVPCSTSHLAVCE
FPI

or a sequence having at least 80%, 85%, 90%, 92%, 94%, 95%, 96%, 97%, 98% or 99% identity thereto.

In one embodiment, the effector protein comprises:

(SEQ ID NO: 42)
MWWRLWWLLLLLLLLWPMVWANITRFQTLLALHRSYLTPGDSSSGWTAG
AAAYYVGYLQPRTFLLKYNENGTITDAVDCALDPLSETKCTLKSFTVEK
GIYQTSNFRVQPTESIVRFPNITNLCPFGEVFNATRFASVYAWNRKRIS
NCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFR
KSNLKPFERDISTEIYQAGSTPCNGVEGFNCYFPLQSYGFQPTNGVGYQ
PYRVVVLSFELLHAPATV

or a sequence having at least 80%, 85%, 90%, 92%, 94%, 95%, 96%, 97%, 98% or 99% identity thereto.

In one embodiment, the effector protein comprises:

(SEQ ID NO: 43)
MRIPLVGKDSIESKDIGFTLIHEHLRVFSEAVRQQWPHLYNEDEEFRN
AVNEVKRAMQFGVKTIVDPTVMGLGRDIRFMEKVVKATGINLVAGTGI
YIYIDLPFYFLNRSIDEIADLFIHDIKEGIQGTLNKAGFVKIAADEPG
ITKDVEKVIRAAAIANKETKVPIITHSNAHNNTGLEQQRILTEEGVDP
GKILIGHLGDTDNIDYIKKIADKGSFIGLDRYGLDLFLPVDKRNETTL
RLIKDGYSDKIMISHDYCCTIDFGTAKPEYKPKLAPRWSITLIFEDTI
PFLKRNGVNEEVIATIFKENPKKFFS

or a sequence having at least 80%, 85%, 90%, 92%, 94%, 95%, 96%, 97%, 98% or 99% identity thereto.

In one embodiment, the effector protein comprises:

(SEQ ID NO: 44)
FKRIVQRIKDFLRRKRFKRTVQRIKDFLRRKRFKRTVQRIKDFLRRKR
FKRIVQRIKDFLRRKRFKRIVQRIKDFLRRKR*

or a sequence having at least 80%, 85%, 90%, 92%, 94%, 95%, 96%, 97%, 98% or 99% identity thereto.

In one embodiment, the effector protein comprises:

(SEQ ID NO: 45)
MGVHECPAWLWLLLSLLSLPLGLPVLGAPPRLICDSRVLERYLLEAKEA
ENITTGCAEHCSLNENITVPDTKVNFYAWKRMEVGQQAVEVWQGLALLS
EAVLRGQALLYNSSQPWEPLQLHVDKAVSGLRSLTTLLRALGAQKEAIS
PPDAASAAPLRTITADTFRKLFRVYSNFLRGKLKLYTGEACRTGDR

or a sequence having at least 80%, 85%, 90%, 92%, 94%, 95%, 96%, 97%, 98% or 99% identity thereto.

In one embodiment, the effector protein comprises:

(SEQ ID NO: 46)
RVQPTESTVRFPNITNLCPFDEVFNATRFASVYAWNRKRISNCVADYSVL
YNLAPFFTFKCYGVSPTKLNDLCFTNVYADSFVIRGDEVRQIAPGQTGNI
ADYNYKLPDDFTGCVIAWNSNKLDSKVSGNYNYLYRLFRKSNLKPFERDI
STEIYQAGNKPCNGVAGFNCYFPLRSYSFRPTYGVGHQPYRVVVLSFELL
HAPATVCGPKKSTNLVKNKCVNF

or a sequence having at least 80%, 85%, 90%, 92%, 94%, 95%, 96%, 97%, 98% or 99% identity thereto.

For any of SEQ ID Nos. 38-46 if present, a native signal peptide may be removed or replaced with a heterologous signal peptide (see below for exemplary signal peptides).

rIAV Strain Generation

HEK293T cells are co-transfected with constructs to generate both vRNA and mRNA species for each of the 8 IAV segments. For generation of rIAV, one or more segments have the protein coding region of the vRNA modified to remove the capability of producing a fully functional viral particle, potentially encoding an alternative effector protein. The missing IAV protein is supplied through an additional construct separately expressing the relevant mRNA. Dual expression pDZ-plasmids express negative strand vRNA of IAV segments as well as positive strand mRNA from the same segments to produce the relevant protein products and are used to supply the unmodified segments and proteins.

The HEK293 Ts are co-cultured with MDCK cells engineered to express the missing protein. The cells are incubated with L-1-tosylamido-2-phenylethyl chloromethyl ketone treated trypsin at 1 ug/mL to aid in viral particle maturation. Viral particles generated in the HEK293 Ts infect the MDCKs where they propagate. When about 70% of the cells have died the supernatant is collected and stored at −80° C.

Exemplary Strains

TABLE 2

| Strain | Effector | Utility |
|---|---|---|
| iAS001 | GFP | Used as a control. |
| iAS002 | SARS-CoV-2-S RBD | Development of antigen tests, |
| | | research |
| iAS003 | Soluble ACE2 receptor | Post-exposure therapy, possibly |
| | | pre-exposure prophylactic |
| iAS004 | Anti-SARS-CoV-2-S ScFv | Post-exposure therapy, possibly |
| | | pre-exposure prophylactic |
| iAS005 | Mannan Binding Lectin | Complement system protein, |
| | | replacement therapy |
| iAS006 | SARS-CoV-1-S RBD | Development of antigen tests, |
| | | research |
| iAS007 | SsoPox W263F | Organophosphate Degradation/ |
| | | post-exposure treatment |
| iAS008 | Lysostaphin | S. aureus antibiotic/post-exposure |
| | | treatment for drug resistant |
| | | variants/cell culture treatment |
| iAS010 | FK-13 | Broad spectrum antibiotic |
| iAS015 | Erythropoietin | Large scale manufacturing, |
| | | Protein therapy for anemia |
| iAS016 | SARS-CoV-2-S RBD | Development of antigen tests, |
| | Omicron Varient | research |

### Exemplary Signal Peptides

In one embodiment, the open reading frame for the effector protein (e.g., anti-microbial) is linked to a leader sequence, e.g., a secretion signal. Exemplary signal peptides are shown in Table 1 below:

TABLE 1

| Leader Sequence Name | Sequence |
|---|---|
| Human OSM | MGVLLTQRTLLSLVLALLFPSMASM (SEQ ID NO: 11) |
| VSV-G | MKCLLYLAFLFIGVNC (SEQ ID NO: 12) |
| Mouse Ig Kappa | METDTLLLWVLLLWVPGSTGD (SEQ ID NO: 13) |
| Human IgG2 H | MGWSCIILFLVATATGVHS (SEQ ID NO: 15) |
| BM40 | MRAWIFFLLCLAGRALA (SEQ ID NO: 16) |
| Secrecon | MWWRLWWLLLLLLLLWPMVWA (SEQ ID NO: 17) |
| Human IgK VIII | MDMRVPAQLLGLLLLWLRGARC (SEQ ID NO: 18) |
| CD33 | MPLLLLLPLLWAGALA (SEQ ID NO: 19) |
| tPA | MDAMKRGLCCVLLLCGAVFVSPS (SEQ ID NO: 20) |
| Human Chymotrypsinogen | MAFLWLLSCWALLGTTFG (SEQ ID NO: 21) |
| Human trypsinogen-2 | MNLLLILTFVAAAVA (SEQ ID NO: 22) |
| Human IL-2 | MYRMQLLSCIALSLALVTNS (SEQ ID NO: 23) |
| Gaussia luc | MGVKVLFALICIAVAEA (SEQ ID NO: 24) |
| Albumin (HSA) | MKWVTFISLLFSSAYS (SEQ ID NO: 25) |
| Influenza Haemagglutinin | MKTIIALSYIFCLVLG (SEQ ID NO: 26) |
| Human insulin | MALWMRLLPLLALLALWGPDPAAA (SEQ ID NO: 51) |
| Silkworm Fibroin LC | MKPIFLVLLVVTSAYA (SEQ ID NO: 28) |

In one embodiment, a signal peptide includes one with a sequence that has at least 80%, 85%, 90%, 92%, 94%, 95%, 96%, 97%, 98% or 99% identity with one of SEQ ID Nos. 11-28.

### Exemplary Targeting Ligands

To enhance targeting to specific tissues or cells, the non-integrating recombinant viruses may include a heterologous extracellular or transmembrane protein including but not limited to proteins and glycoproteins such as VSV-G, or proteins from LCMV (e.g., to target pancreatic islet cells, Liver, central nervous system, or cancer cells), RRV proteins, e.g., to target liver, SeV F, e.g., to target liver or lung, Ebola virus proteins to target, e.g., lung, including Marburg virus proteins, Rabies virus or Mokola virus proteins to target the central nervous system, RD114 or GALV proteins to target the hematopoietic system.

### Exemplary Viral Delivery Vectors

In one embodiment, the recombinant virus is an influenza virus, e.g., a replication defective influenza virus. For example, for influenza viruses with 8 viral segments, one of the viral segments is modified to include sequences for the protein antibiotic. The modification may be an insertion into a coding region of the viral segment, which insertion disrupts (e.g., prevents) expression of the viral protein encoded by the coding region. The modification may include a deletion of a coding region of the viral segment, which disrupts (e.g., prevents) expression of the viral protein encoded by the coding region. The modification may include one or more nucleotide substitutions in a coding region of the viral segment which substitutions may result in altered reading frame or a stop codon, thereby disrupting (e.g., prevents) expression of a functional viral protein encoded by the coding region). The viral protein that is not capable of being expressed by one of the viral segments may be provided in trans when preparing the recombinant virus.

In one embodiment, the recombinant virus is an isavirus, e.g., a replication defective isavirus. For example, one of the 10 viral segments is modified to include sequences for the protein antibiotic. The modification may be an insertion into a coding region of the viral segment, which insertion disrupts (e.g., prevents) expression of the viral protein encoded by the coding region. The modification may include a deletion of a coding region of the viral segment, which disrupts (e.g., prevents) expression of the viral protein encoded by the coding region. The modification may be one or more nucleotide substitutions in a coding region of the viral segment which substitutions may result in altered reading frame or a stop codon, thereby disrupting (e.g., prevents) expression of a functional viral protein encoded by the coding region). The viral protein that is not capable of being expressed by one of the viral segments may be provided in trans when preparing the recombinant virus.

In one embodiment, the recombinant virus is a quaranjavirus, e.g., a replication defective quaranjavirus. For example, one of the 6 viral segments is modified to include sequences for the protein antibiotic. The modification may be an insertion into a coding region of the viral segment, which insertion disrupts (e.g., prevents) expression of the viral protein encoded by the coding region. The modification may include a deletion of a coding region of the viral segment, which disrupts (e.g., prevents) expression of the viral protein encoded by the coding region. The modification may be one or more nucleotide substitutions in a coding region of the viral segment which substitutions may result in altered reading frame or a stop codon, thereby disrupting (e.g., prevents) expression of a functional viral protein encoded by the coding region). The viral protein that is not capable of being expressed by one of the viral segments may be provided in trans when preparing the recombinant virus.

In one embodiment, the recombinant virus is a thogotavirus, e.g., a replication defective thogotaavirus. For example, one of the 6 or 7 viral segments is modified to include sequences for the protein antibiotic. The modification may be an insertion into a coding region of the viral segment, which insertion disrupts (e.g., prevents) expression of the viral protein encoded by the coding region. The modification may include a deletion of a coding region of the viral segment, which disrupts (e.g., prevents) expression of the viral protein encoded by the coding region. The modification may be one or more nucleotide substitutions in a coding region of the viral segment which substitutions may result in altered reading frame or a stop codon, thereby disrupting (e.g., prevents) expression of a functional viral protein encoded by the coding region). The viral protein that is not capable of being expressed by one of the viral segments may be provided in trans when preparing the recombinant virus.

Baceteriophage that are generated from, for example, animal cells such as mammalian cells, may also be employed. For instance, positive-sense single-stranded RNA phage may be employed to deliver a protein antibiotic. Exemplary phage include but are not limited to MS2 phage, bacteriophage f2, bacteriophage Qβ or any of the Leviviridae family of phages. As described above, the phage genome is modified to include an open reading frame for the protein antibiotic and in one embodiment, at least one of the phage proteins may be modified to reduce or eliminate expression.

### EXEMPLARY EMBODIMENTS

In one embodiment, the disclosure provides an isolated replication defective influenza virus comprising a genome where at least one of the viral segments comprises a heterologous nucleotide sequence comprising a nucleotide segment that encodes an effector protein (e.g., an antimicrobial protein). The replication defective influenza virus is infectious in the sense that it infects cells and the viral segments are capable of being replicated and/or transcribed, however, the genetic content of the virus does not allow for subsequent rounds of infection (a “single cycle virus”), e.g., the infection does not result in progeny virus as the genetic content is deficient in one or more proteins that are needed for progeny virus production. In one embodiment, at least one of the regions on a viral segment that code for an influenza virus protein is modified so that after infection, a functional form of the viral protein cannot be produced from the viral segment. The modification can include an insertion of one or more nucleotides, a deletion of one or more nucleotides, or a substitution of one or more nucleotides. The site of that modification may also be the site of insertion of the heterologous sequence. For example, the heterologous sequence may replace sequence that code for HA or any of the other viral proteins. In one embodiment, the virus is an influenza A virus. In one embodiment, the virus is an influenza B virus. In one embodiment, the heterologous nucleotide sequence comprises a secretory signal sequence operably linked to the nucleotide segment. In one embodiment, the protein is an antibacterial protein. In one embodiment, the protein is a heterologous antiviral protein. In one embodiment, the protein is an antifungal protein. In one embodiment, the antimicrobial protein comprises lysostaphin. In one embodiment, the genome of the virus further comprises a heterologous nucleotide segment that encodes targeting ligand. In one embodiment, the virus further comprises a targeting ligand. In one embodiment, the HA viral segment comprises the heterologous nucleotide sequence. In one embodiment, the HA coding sequence in the HA viral segment is disrupted so as not to encode a functional HA. In one embodiment, the virus is an influenza virus comprising HAL HA2 and NA on its surface. In one embodiment, the virus is an influenza virus comprising a heterologous glycoprotein on its surface but which may lack HA1 and/or HA2 and/or NA. In one embodiment, the virus is an influenza virus comprising PA, PB1, PB2, NP, M1, M2, NS1, NS2, or any combination thereof.

Further provided is a method to prevent, inhibit or treat microbial infection in an animal, comprising: administering to the animal an effective amount of a composition comprising the isolated replication defective influenza virus. In one embodiment, the composition is locally delivered. In one embodiment, the composition is systemically delivered. In one embodiment, the composition is intranasally delivered. In one embodiment, the composition is topically delivered. In one embodiment, the animal is an avian. In one embodiment, the animal is a mammal, e.g., a human, bovine, equine, swine, ovine, caprine, feline or canine.

Further provided is a method to prevent, inhibit or treat heterologous viral infection in an animal, comprising: administering to the animal an effective amount of a composition comprising the isolated replication defective influenza virus. In one embodiment, the composition is locally delivered. In one embodiment, the composition is systemically delivered. In one embodiment, the composition is intranasally delivered. In one embodiment, the composition is topically delivered. In one embodiment, the animal is an avian. In one embodiment, the animal is a mammal, e.g., a human, bovine, equine, swine, ovine, caprine, feline or canine.

Further provided is a method to degrade or modify chemical compounds in an animal, comprising: administering to the animal an effective amount of a composition comprising the isolated replication defective influenza virus. In one embodiment, the composition is locally delivered. In one embodiment, the composition is systemically delivered. In one embodiment, the composition is intranasally delivered. In one embodiment, the composition is topically delivered. In one embodiment, the animal is an avian. In one embodiment, the animal is a mammal, e.g., a human, bovine, equine, swine, ovine, caprine, feline or canine.

Further provided is a method to biomanufacture protein products in an unengineered cell line, comprising: administering to the cell line an effective amount of a composition comprising the isolated replication defective influenza virus. In one embodiment, the cell line is an avian. In one embodiment, the cell line is mammalian, e.g., a human, bovine, equine, swine, ovine, caprine, feline or canine.

Further provided is a method to supplement endogenous protein production in an animal, comprising: administering to the animal an effective amount of a composition comprising the isolated replication defective influenza virus. In one embodiment, the composition is locally delivered. In one embodiment, the composition is systemically delivered. In one embodiment, the composition is intranasally delivered. In one embodiment, the composition is topically delivered. In one embodiment, the animal is an avian. In one embodiment, the animal is a mammal, e.g., a human, bovine, equine, swine, ovine, caprine, feline or canine.

### Pharmaceutical Compositions

Pharmaceutical compositions, suitable for inoculation, e.g., nasal, parenteral or oral administration, comprise one or more virus or phage isolates, e.g., influenza virus isolates, optionally further comprising sterile aqueous or non-aqueous solutions, suspensions, and emulsions. The compositions can further comprise auxiliary agents or excipients, as known in the art. The composition is generally presented in the form of individual doses (unit doses).

Conventional compositions generally contain about 0.1 to 200 e.g., 30 to 100 μg, of a major viral glycoprotein, e.g., influenza HA, from each of strain in the composition. The the main constituent of the composition may comprise a single virus, e.g., a single influenza virus, or a combination of viruses, for example, at least two or three influenza viruses, including one or more reassortant(s).

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition of the present invention is used for administration to an individual, it can further comprise salts, buffers, adjuvants, or other substances which are desirable for improving the efficacy of the composition. Adjuvants, substances which can augment a specific immune response, can be used. Normally, the adjuvant and the composition are mixed prior to presentation to the immune system, or presented separately, but into the same site of the organism being immunized.

Heterogeneity in a composition may be provided by mixing viruses, e.g., at least two influenza virus strains, such as 2-20 strains or any range or value therein. Compositions can be provided for variations in a single strain of a virus, using techniques known in the art.

A pharmaceutical composition according to the present invention may further or additionally comprise at least one chemotherapeutic compound, for example, for gene therapy, immunosuppressants, anti-inflammatory agents or immune enhancers, as well as chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition may be for either a “prophylactic” or “therapeutic” purpose. When provided prophylactically, the composition may be provided before any symptom or clinical sign of a pathogen infection becomes manifest. The prophylactic administration of the composition serves to prevent or attenuate any subsequent infection. When provided therapeutically, the composition may be provided upon the detection of a symptom or clinical sign of actual infection. The therapeutic administration of the composition(s) serves to attenuate any actual infection. Thus, a composition of the present invention may be provided either before the onset of infection (so as to prevent or attenuate an anticipated infection) or after the initiation of an actual infection.

A composition is said to be “pharmacologically acceptable” if its administration can be tolerated by a recipient mammal. Such an agent is said to be administered in a “therapeutically effective amount” if the amount administered is physiologically significant. A composition of the present invention is physiologically significant if its presence results in a detectable change in the physiology of a recipient patient.

The “protection” provided need not be absolute, i.e., the infection need not be totally prevented or eradicated, if there is a statistically significant improvement compared with a control population or set of mammals. Protection may be limited to mitigating the severity or rapidity of onset of symptoms or clinical signs of the influenza virus infection.

### Pharmaceutical Administration

A composition having the recombinant virus may prevent, inhibit or treat infection by one or more microbial pathogens, e.g., one or more bacterial strains, one or more fungal strains or one or more heterologous viral strains.

The present invention thus includes methods for preventing or attenuating an infection by at least one strain of pathogen. As used herein, a composition is said to prevent or attenuate an infection if its administration results either in the total or partial attenuation (i.e., suppression) of a clinical sign or condition of the infection.

Further provided is a composition having the recombinant virus may prevent, inhibit, or treat, or modify effects of chemical compounds in an animal. As used herein, a composition is said to prevent or attenuate the effects of a chemical compound if its administration results either in the total or partial attenuation (i.e., suppression) of a clinical sign or condition of the compound.

Further provided is a composition having the recombinant virus be used for in vitro biomanufacturing of proteins of interest.

Further provided is a composition having the recombinant virus be used to supplement in vivo production of proteins.

A composition having at least one non-integrating virus of the present invention, including one which is attenuated, e.g., replication defective, and one or more other isolated viruses, one or more isolated viral proteins thereof, one or more isolated nucleic acid molecules encoding one or more viral proteins thereof, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating a pathogen related pathology, comprises administration of an effective amount of a composition as described herein, administered as a single treatment, or repeated as enhancing or booster dosages, over a period up to and including between one week and about 24 months, or any range or value therein.

According to the present disclosure, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the invention and represent dose ranges.

The dosage of a virus, e.g., an influenza virus, for an animal such as a mammalian adult organism may be from about 10²-10¹⁵, e.g., 10³-10¹², plaque forming units (PFU)/kg, or any range or value therein. The dose may range from about 0.1 μg to 1000 μg, e.g., 30 μg to 100 μg, of HA protein. However, the dosage should be a safe and effective amount as determined by conventional methods. The dosage of immunoreactive HA in each dose of virus may be standardized to contain a suitable amount, e.g., 30 μg to 100 μg or any range or value therein, or the amount recommended by government agencies or recognized professional organizations. The quantity of influenza NA can also be standardized, however, this glycoprotein may be labile during purification and storage.

The dosage of immunoreactive HA in each dose of virus can be standardized to contain a suitable amount, e.g., 1-50 μg or any range or value therein, e.g., 15 μg per component for older children (greater than or equal to 3 years of age), and 7.5 μg per component for children less than 3 years of age. The quantity of NA can also be standardized, however, this glycoprotein can be labile during the processor purification and storage. Each 0.5 ml dose may contain approximately 1-50 billion virus particles, and preferably 10 billion particles.

### Certain Embodiments

In certain embodiments, the present invention provides an isolated replication defective non-integrating segmented virus comprising a genome where at least one of the viral segments comprises a heterologous nucleotide sequence comprising a nucleotide segment that encodes an effector protein.

In certain embodiments, the virus is an influenza virus, which comprises an HA viral segment.

In certain embodiments, the heterologous nucleotide sequence comprises a secretory signal sequence operably linked to the nucleotide segment.

In certain embodiments, the signal sequence has at least 80% amino acid sequence identity to one of SEQ ID Nos. 11-28.

In certain embodiments, the effector protein is an antimicrobial protein.

In certain embodiments, the antimicrobial protein lyses bacteria.

In certain embodiments, the bacteria include Staphylococcus.

In certain embodiments, the antimicrobial protein comprises lysostaphin.

In certain embodiments, the antimicrobial protein comprises a sequence having at least 80% amino acid sequence identity to one of SEQ ID Nos. 1-10 or 29-37, or a portion thereof with antimicrobial activity.

In certain embodiments, the effector protein comprises a sequence having at least 80% amino acid sequence identity to one of SEQ ID Nos. 38-46, or a portion thereof with biological activity.

In certain embodiments, the effector protein is an SsoPox protein,

In certain embodiments, the effector protein is a SARS-CoV-2 spike protein.

In certain embodiments, the effector protein is SARS-CoV-2 Spike Receptor Binding Domain.

In certain embodiments, the effector protein is an Anti-SARS-CoV-2-ScFv

In certain embodiments, the effector protein SARS-CoV-2 Omicron Spike Receptor Binding Domain.

In certain embodiments, the effector protein is a ACE2 Receptor.

In certain embodiments, the effector protein is Mannan-binding lectin.

In certain embodiments, the effector protein FK-13.

In certain embodiments, the effector protein Erythropoietin.

In certain embodiments, the genome further comprises a heterologous nucleotide segment that encodes a heterologous targeting ligand.

In certain embodiments, the HA viral segment comprises the heterologous nucleotide sequence.

In certain embodiments, the HA coding sequence in the HA viral segment is disrupted so as not to encode a functional HA.

In certain embodiments, the present invention provides method to prevent, inhibit or treat microbial infection in an animal, comprising administering to the animal an effective amount of a composition comprising an isolated replication defective non-integrating segmented virus comprising a genome where at least one of the viral segments comprises a heterologous nucleotide sequence comprising a nucleotide segment that encodes an effector protein.

In certain embodiments, the composition is delivered locally, systemically, intranasally, or topically.

In certain embodiments, the animal is an avian.

In certain embodiments, the animal is a mammal.

In certain embodiments, the animal is a human.

In certain embodiments, the animal has a bacterial infection.

In certain embodiments, the mammal has a Staphylococcus, Proteus, Listeria, or Pseudomonas infection.

In certain embodiments, the animal has a fungal infection.

In certain embodiments, the antimicrobial protein comprises a cutonase-like serine esterase, defensin, indolicidin, protegrain, LL-37, S-type pyocin or tailocin.

The invention will be described by the following non-limiting examples.

### Example 1

### Materials and Methods

### Generating Lysostaphin Expression Plasmid

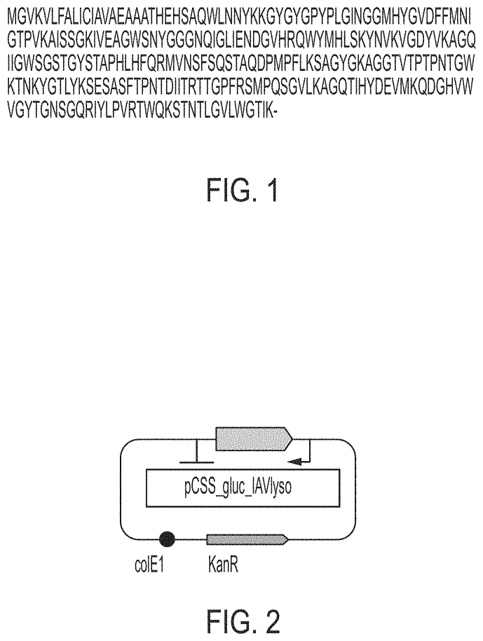

The lysostaphin encoding plasmid was generated using, a gBlock synthesized by IDT (Coralville, Iowa) encoding the lysostaphin mature peptide with Gln125,232 mutations (based on Kerr, D. E. et al. Nat. Biotechnol. 19, 66 (2001)) and the gaussia luciferase secretion signal, see FIG. 1 for gluc_lyso amino acid sequence. To generate the pCSS_gluc_IAVlyso plasmid for influenza A virus mediated expression and viral packaging, the lysostaphin gBlock was amplified using primers 5′-atgcaCACCTGCTACTtcaCTTAATGGTGCCCCACAGTAC-3′ (SEQ ID NO: 47) and 5′-TGCATCACCTGCCCATATGGGCGTGAAGGTCCTGTTCG-3′ (SEQ ID NO: 48) and the destination plasmid was amplified from pCSS_IAV1 plasmid template using primers 5′-atgcaCACCTGCTACTCCATGGCTAGCCTATACAAATTGTGTCTGCAACTGC (SEQ ID NO: 49) and 5′-TGCATCACCTGCCCATGTGACTCGAGATCTACTCAACTGTCGCCAGTTC-3′ (SEQ ID NO: 50). The resulting PCR products were assembled via a one-pot Aar I restriction digestion-ligation reaction (GeneArt™ Type IIs Assembly Kit, Aar I ThermoFisher Scientific). The resulting lysostaphin expression construct contains 5′ and 3′ ends of influenza A virus (IAV) RNA dependent RNA polymerase recognition and virus packaging sequences from the hemagglutinin (HA) IAV gene. Expression of the gene is driven by a human polymerase-I promoter (FIG. 2).

Amino acid sequence of gluc_lyso:
(SEQ ID NO: 1)
MGVKVLFALICIAVAEAAATHEHSAQWLNNYKKGYGYGPYPLGINGGMH
YGVDFFMNIGTPVKAISSGKIVEAGWSNYGGGNQIGLIENDGVHRQWYM
HLSKYNVKVGDYVKAGQIIGWSGSTGYSTAPHLHFQRMVNSFSQSTAQD
PMPFLKSAGYGKAGGTVTPTPNTGWKTNKYGTLYKSESASFTPNTDIIT
RTTGPFRSMPQSGVLKAGQTIHYDEVMKQDGHVWVGYTGNSGQRIYLPV
RTWQKSTNTLGVLWGTIK-

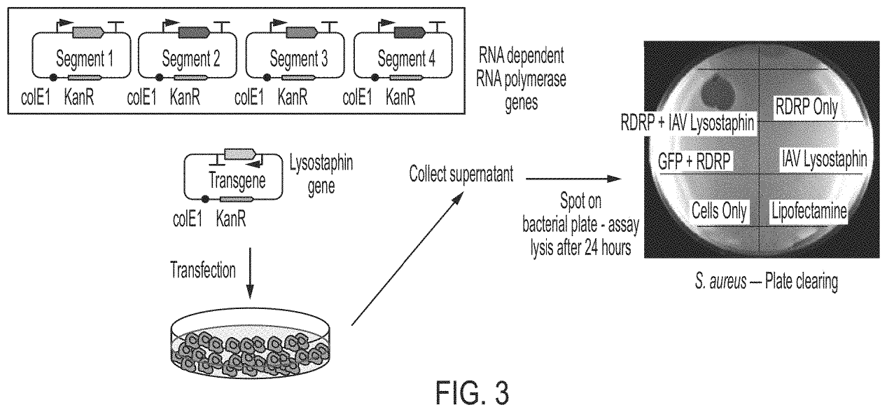

### Generating Lysostaphin Protein Using the RNA Dependent RNA Polymerase (RDRP) of IAV (FIG. 3)

To isolate secreted lysostaphin for activity assays, pDZ plasmids encoding the nucleoprotein (NP), RDRP proteins (described previously in Quinlivan, M. et al. J. Virol. 79, 8431-8439 (2005)) and pCSS_gluc_IAVlyso were transfected into human embryonic kidney 293T cells as follows. Each plasmid was diluted to achieve a concentration of 0.5 ┌g of each plasmid in a total volume of 50 μl in OptiMEM (GIBCO, Invitrogen). Then 5 ┌L of lipofectamine 2000 was added and the mixture was incubated for 30 mins at room temperature before being added to into one well of a 6-well plate with human embryonic kidney 293T (HEK293T) cells at 70% confluence in Dulbecco's modified Eagle's medium (DMEM) supplemented with 10% FBS. Supernatant was harvested after 48 hours and stored at −80° C.

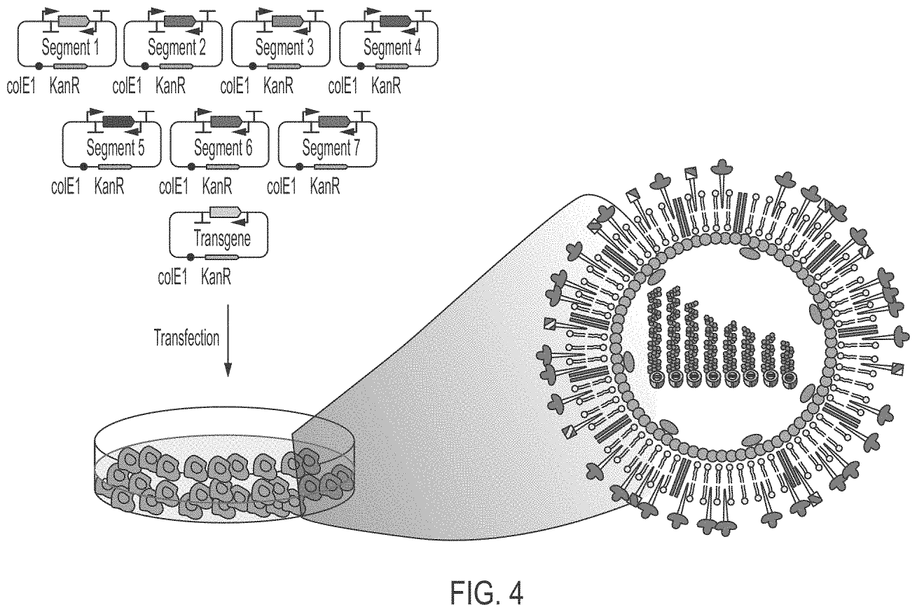

### Virus Rescue (FIG. 4).

Viruses were rescued in 293T cells by plasmid-based transfection with IAV PR8 in the pDZ vector using methods previously described (Hoffmann E, Neumann G, Kawaoka Y, Hobom G, & Webster R G. PNAS 97(11):6108-6113 (2000)). To generate recombinant single cycle viruses, the pDZ-HA plasmid was replaced with pCSS_gluc_IAVlyso. pCAGGs WSN HA plasmid was supplemented into the transfection. 24 h following transfection, 750,000 MDCK WSN HA cells were added to the culture in Opti-MEM containing TPCK trypsin (0.5 ug/mL). The following day, 500 uL Opti-MEM containing TPCK trypsin (1 ug/mL) was added. The next day, 500 uL Opti-MEM containing TPCK trypsin (2 ug/mL) was added to the culture. One day later, the supernatant was harvested, centrifuged to remove cellular debris, and stored at −80° C.

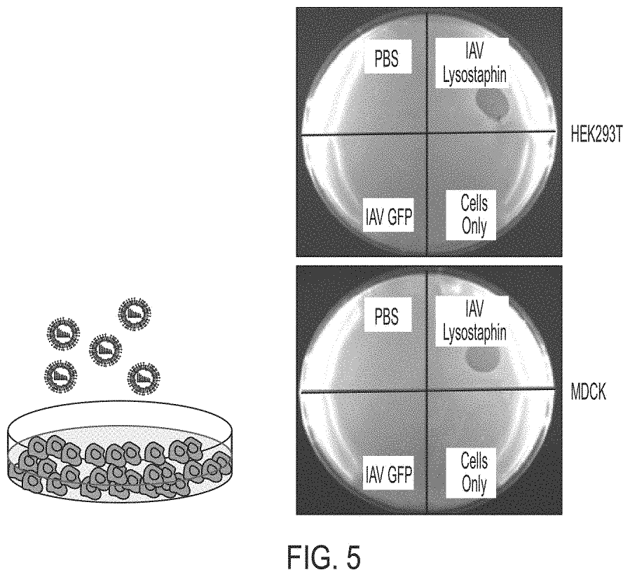

Generating Lysostaphin Protein Through Infection of Cells with gluc_IAVlyso Virus (FIG. 5).

HEK293T and Madin-Darby canine kidney (MDCK) cells were infected with an MOI of 0.6 to determine ability of recombinant lysostaphin virus to generate functional protein. Infections were carried out in infection media (PBS with 10% Ca/Mg, 1% pen/strep, 5% BSA) at 37° C. for 1 h. Infection media was then replaced with (DMEM) supplemented with 10% FBS. The supernatant was harvested after 48 hours

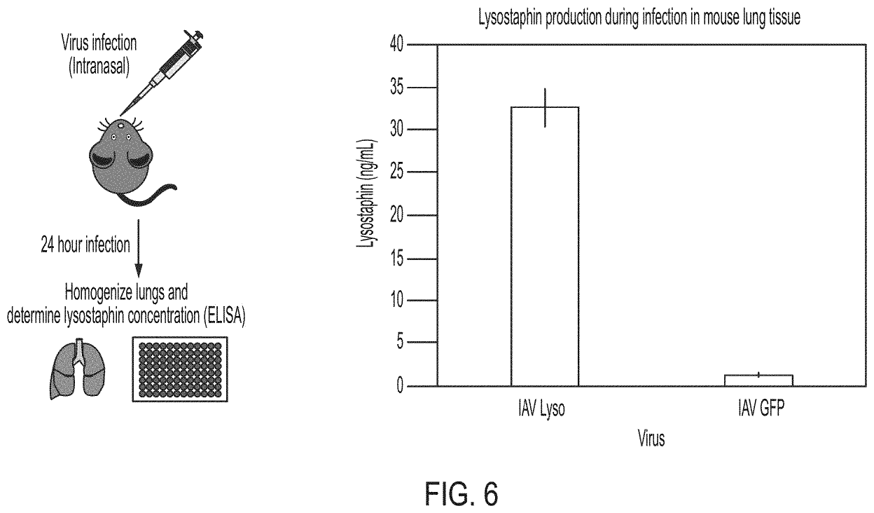

### In Vivo Production of Lysostaphin in Mouse Lung Tissue (FIG. 6).

Ten-week-old female C57BL/6 mice (Jackson Laboratories) were sedated with ketamine/xylazine and infected intranasally with 50 μl of 10⁵ PFU of gluc_IAVlyso influenza recombinant virus. GFP recombinant virus was included as a control. Mice were sacrificed 24 hours after infection. The lungs were removed, homogenized and lysostaphin concentration was determine by ELISA.

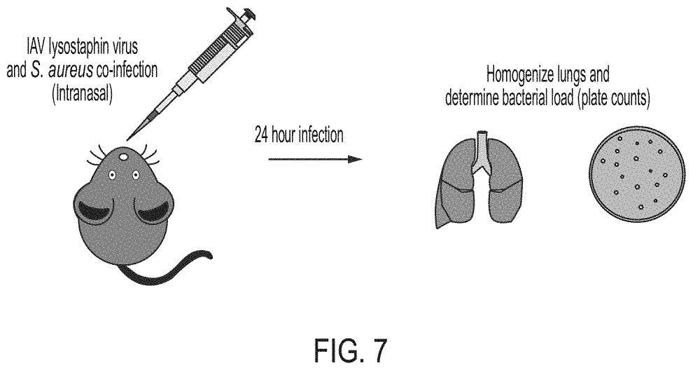

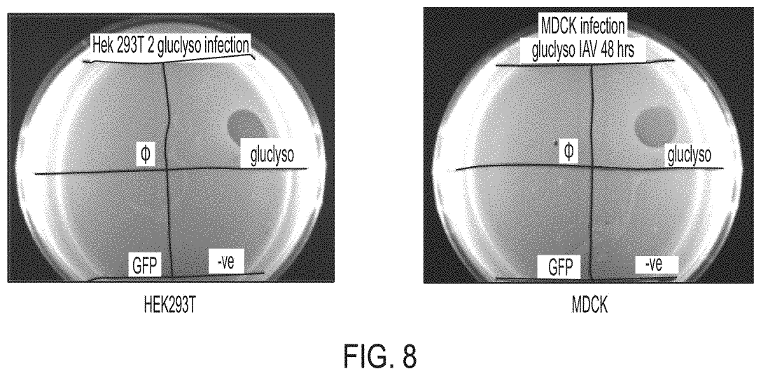

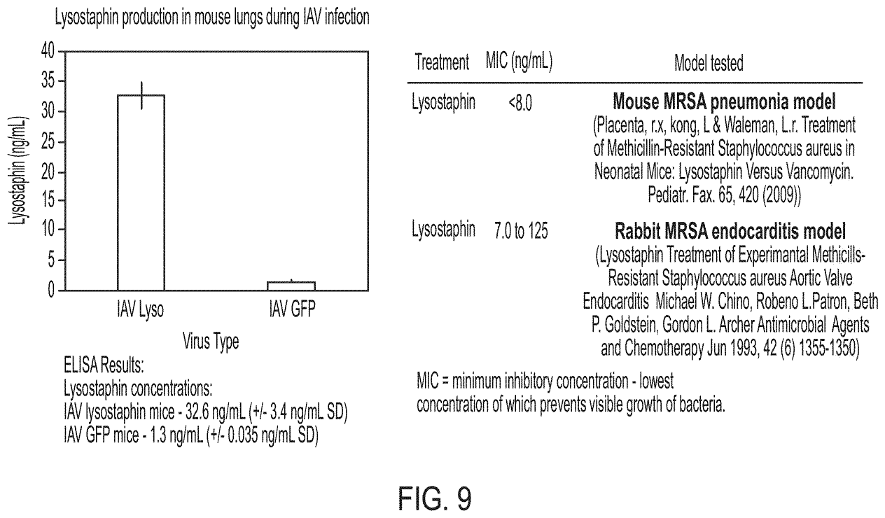

Treatment of MRSA Pneumonia with IAV Lysostaphin Expressing Virus (FIG. 7).

Mice were sedated with ketamine/xylazine and infected intranasally with 50 μl of 10⁵ PFU of gluc_IAVlyso (IAV Lyso) or GFP (IAV GFP) influenza recombinant virus. PBS or recombinant lysostaphin were used as controls. Mice were infected with MRSA intranasally with 50 μl of 2.4×10⁴ CFU. Mice were sacrificed after 24 or 48 hours, serum was callected, lungs were homogenized and plated to determine CFU in treated and untreated mice.

### Results

### Generating Lysostaphin Construct and Successful Expression of Secreted Lysostaphin.

The assembled gluc signal sequence and mature peptide sequence of lysostaphin containing mature peptide with Gln125,232 mutations were successfully assembled into the pCSS_IAV1 backbone generating pCSS_gluc_IAVlyso (FIG. 2). The constructs were sequence verified and tested for expression using the IAV RDRP complex in HEK293T cells. The resulting supernatant showed clearance on a spot-on plate assay using MRSA indicating that the construct was functional in 2 ways: (i) the gene was expressed and recognized by the IAV RDRP complex, and (ii) the protein was successfully secreted into the media in an active form (FIG. 3).

### Rescue and Functional Assays of Single Cycle IAV Virus Containing the a Lysostaphin Expressing Gene Segment.

The gene fragment comprising the secreted lysostaphin construct was packaged into IAV through transfection and amplification using HEK293T and MDCK WSN HA cells respectively (FIG. 4). The virus was tittered and used to infect confluent HEK293T and MDCK cells. The resulting supernatant was spotted onto plates containing MRSA and demonstrated clearance. This indicated that the lysostaphin construct could be packaged into an infectious virus successfully and used to infect different cell types leading to secretion of lysostaphin (FIG. 5).

### Lysostaphin is Produced in in the Lungs of IAV Lyso Infected Mice.

Mice were infected intranasally with 50 μl of 10⁵ PFU of gluc_IAVlyso (IAV Lyso) or GFP (IAV GFP) influenza recombinant virus to determine production of lysostaphin in the lungs. Lysostaphin specific ELISA indicated that 32.6 ng/mL (+/−3.4 ng/mL SD) was being produced in the lung tissue. This demonstrated that the IAV Lyso recombinant virus was able to infect the cells of the lungs, and those cells were able to produce therapeutically relevant concentration of lysostaphin in the lungs. Concentrations as low as 7 ng/mL have been shown to be effective in treating pneumonia caused by MRSA (Placencia, F. X., Kong, L. & Weisman, L. E. Pediatr. Res. 65, 420 (2009).

### Treatment of MRSA Pneumonia Using Recombinant IAV Lyso Virus.

Mice were infected with intranasally with MRSA and the IAV Lyso recombinant virus to identify ability of the recombinant virus to treat an active infection.

### Example 2

### Transient Gene Delivery Via Domesticated Influenza a Virus

Protein-based therapeutics are increasingly attractive due to their high selectivity and potent efficacy but still suffer from low bioavailability and high manufacturing cost, and requirement for intravenous delivery. Transient RNA-mediated delivery is a safe alternative that allows for expression of protein-based therapeutics within the target cells or tissues but is limited by delivery efficiency. Here, we develop recombinant single-cycle Influenza A Virus as a platform for transient gene delivery in vivo and in vitro for therapeutic, research, and manufacturing applications. The use of this platform serves to produce high-titers of therapeutic protein with a low dosage. Alternatively, high-doses delivered to cell culture induce a binary switch between zero and maximal production of the desired protein product. Finally, we demonstrate the flexibility of our system through rapid repurposing and expression of multiple effector proteins.

Introduction

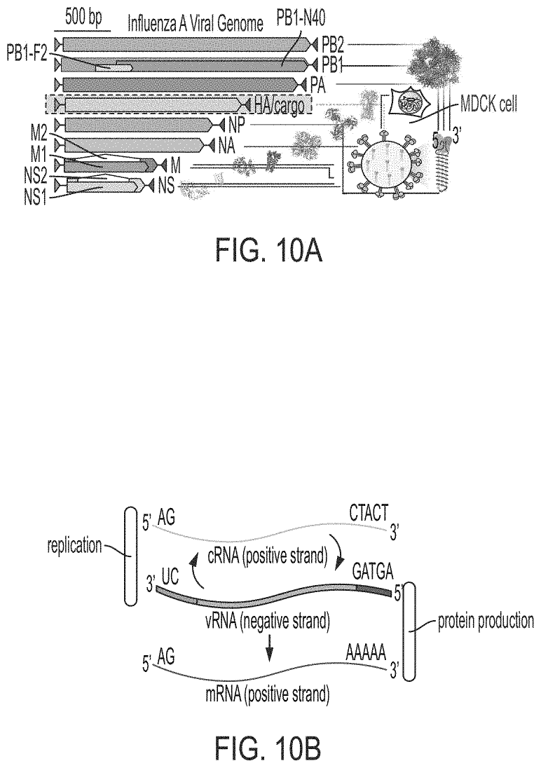

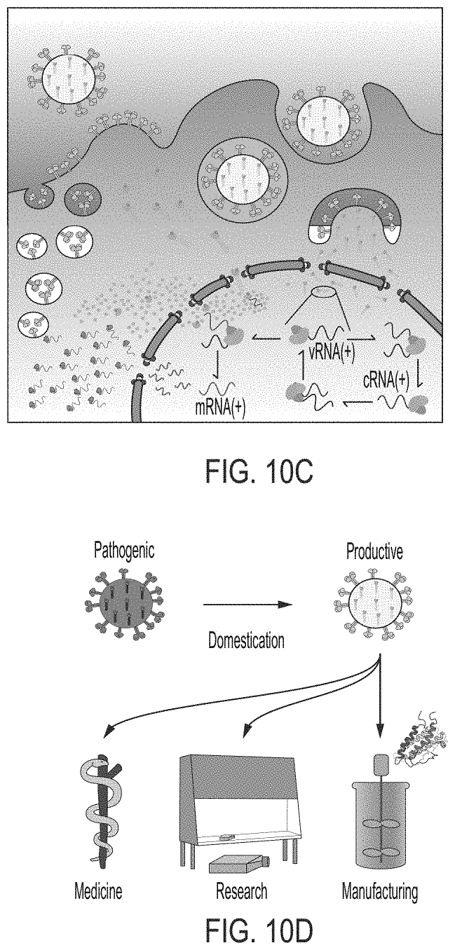

Throughout history, humans have refined and reshaped nature to create tools and technologies with desirable properties. Here, we explore and report several possible applications of domesticated Influenza A Virus (IAV). IAV is a negative sense, single-stranded RNA virus that parasitizes human and other animal cells to replicate and spread (FIG. 10A). In doing so, it causes between 9 and 41 million influenza cases per year in the US. Structurally, IAV particles are composed of an 73-200 nm elliptical lipid capsule stolen from host cells. This capsule is decorated by 400-500 hemagglutinin (HA) and neuraminidase (NA) glycoproteins that are used for viral budding from and entry to host cells. The weak 3 mM affinity of HA to sialic acid means that multivalent binding is likely required for proper attachment to cells. Due to this property, the virus evolved so that the glycoproteins comprise 50% of the particles by weight. Analogous to how predatory behaviors of wolves were co-opted for use in herding via careful breeding, we can coopt IAV's high production of HA for production of human applicable cargo.

Once attached to the cell, the viron is pulled into the cellular endosome where the M2 ion channel reacts to the low pH and releases its interior, namely eight negative (−) strand RNA genomic segments, stored as ribonucleoproteins (RNPs) clustered in a 7+1 arrangement (FIG. 10A-10B). Unlike many other RNA viruses, the viral genome is transported to the nucleus before initiating replication (FIG. 10B). In the nucleus, regulatory sequences unique to each segment are recognized by the RNA-Dependent RNA Polymerase (RDRP) and transcribed from the negative sense vRNA(−) to positive sense mRNA(+) (FIG. 10C). The mRNA(+) is exported to the cytoplasm and translated to generate new viral proteins. Replication of vRNA(−) occurs through a positive sense cRNA(+) intermediate (FIG. 10C). During packaging of the virus, one copy of each segment is packaged into each particle in a highly selective manner that is mediated by segment specific 5′ and 3′ non-coding sequences that flank each vRNA(−). It is important to note that the regulatory segments are independent of the protein coding region allowing us to usurp the endogenous HA (or another segment) for virtually any sequence. In doing so we take two large steps towards domestication of the virus. First, the new strain produces a protein useful to humans, akin to breeding of sheep to produce wool. Second, since the virus can no longer produce HA, we neuter its ability to replicate without human assistance as we have done for silkworms. These two steps fundamentally change the virus from a pathogenic parasite to a productive, domesticate partner.

Compared to ancestral domestication efforts, our process benefits from the short 5-11 hour generation time, the nanometer scale size of our organism, and modern genetic engineering technologies. These features allow us to domesticate the virus for multiple uses in parallel. In this work, we demonstrate the utility of this process for therapeutic, research, and industrial manufacturing applications.

Results

Basic Properties of the System.

New IAV particles can be rescued in cell culture using a pool of 8 (one for each segment) dual expression plasmids that express the vRNA(−) via polymerase I promoters and mRNA(+) via polymerase II promoters. To domesticate the virus, we divide the mRNA(+) and vRNA(−) production and what they encode. Namely, we express a recombinant HA vRNA(−) encoding a protein of interest instead of the endogenous product. When viral particles are produced, our vRNA(−) is packaged. We provide the HA protein necessary to produce active particles through expressing an independent HA mRNA(+). This strategy has been previously used to encode reporters or develop bivalent vaccines.

We initially developed sciIAV strains, expressing either GFP (GFP-sciIAV) or lysostaphin (lyso-sciIAV), a metalloprotease that targets penta-glycine bridges present in the peptidoglycan layer of Staphylococcus spp. bacteria, for characterizing the several fundamental properties of the system. Previous studies of IAV reported “superpromoter” mutations in the 5′ regulatory region of the vRNA(−) that increase the production and expression of the mutant segment. We generated GFP and lysostaphin strains with these mutation (spGFP-sciIAV and splyso-sciIAV respectively).

Strand Specific RT.

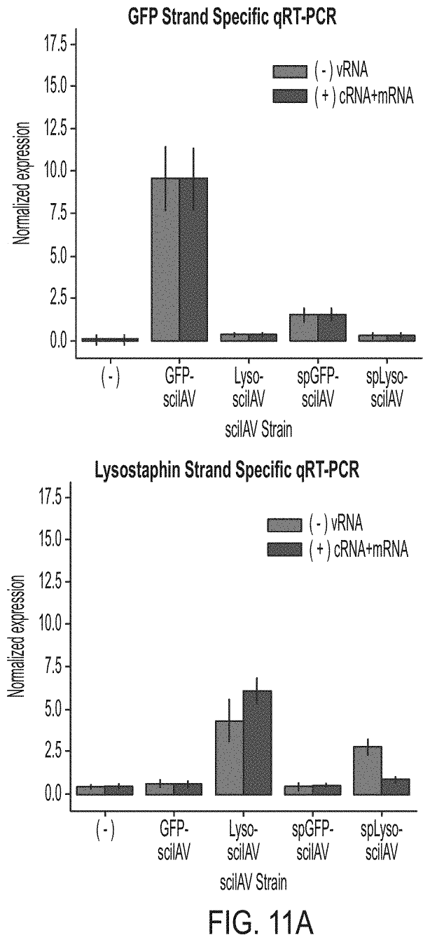

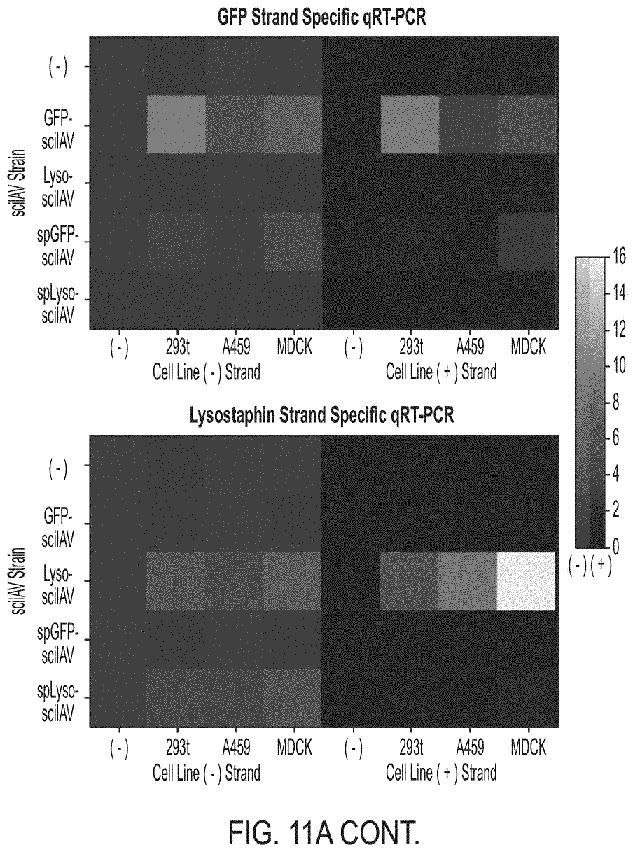

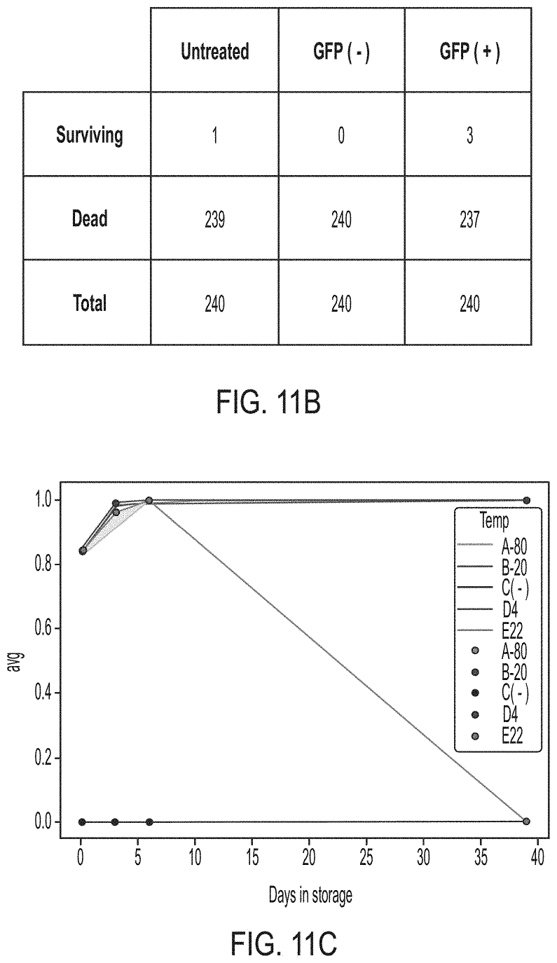

We first wanted to confirm the presence of both (−) and (+) strand RNA species in a range of transduced cells. HEK293t, A549, and wild-type MDCK (MDCK-Wt) cells were transduced with sciIAV carrying either GFP or lysostaphin. After 24 hours, RNA from lysed cells was used to generate cDNA using either cRNA(+)/mRNA(+) or vRNA(−) specific primers. qRT-PCR confirmed high levels of both strands (FIG. 11) in a strain specific manner. The different cell lines ranged in how strongly the RNA species were expressed. Noting that the superpromoter variants performed worse than the natural variant, the remainder of our work focused on reference generated strains. We used RT-PCR with primers targeting the HA regulatory region to amplify the entire fragment for sequencing confirmation.

FACS Cytotoxicity.

Having confirmed the transient nature of our platform, we wanted to determine the mechanism of expression termination (i.e., cytotoxicity or RNA degradation). We transduced HEK293t cells with GFP-sciIAV at 0.05 MOI and used Flow-Assisted Cell Sorting (FACS) to collect single cells. We collected 240 cells each from three populations: GFP positive cells, GFP negative cells, and GFP negative from an untreated population. The clones were allowed to recover. We had low overall survival in all three populations but using Fisher's exact test detected no difference between the populations (FIG. 11).

Stability.

A practical consideration for a transient gene delivery platform is the stability of the viral particles under a variety of storage conditions. We generated a fresh stock of GFP-sciIAV and partitioned it into small 40 μL aliquots stored at either 22° C., 4° C., −20° C., or −80° C. Over a time course, transduced 10⁶ HEK293t cells with 33 μL from each stock (approximately 1.07 MOI initially) and measured the fraction of infected cells. At room temperature the viral particles fully degrade withing 39 days. However, the remaining three conditions maintained 100% infectivity even after 39 days (FIG. 11).

In Vivo Production of Antibacterial Therapeutic Proteins.

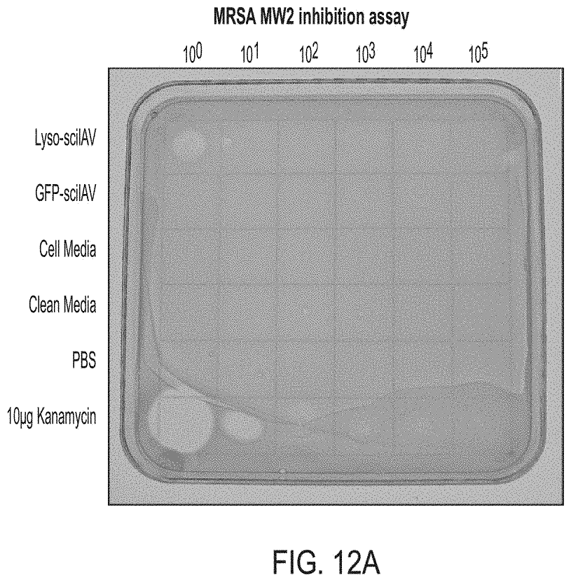

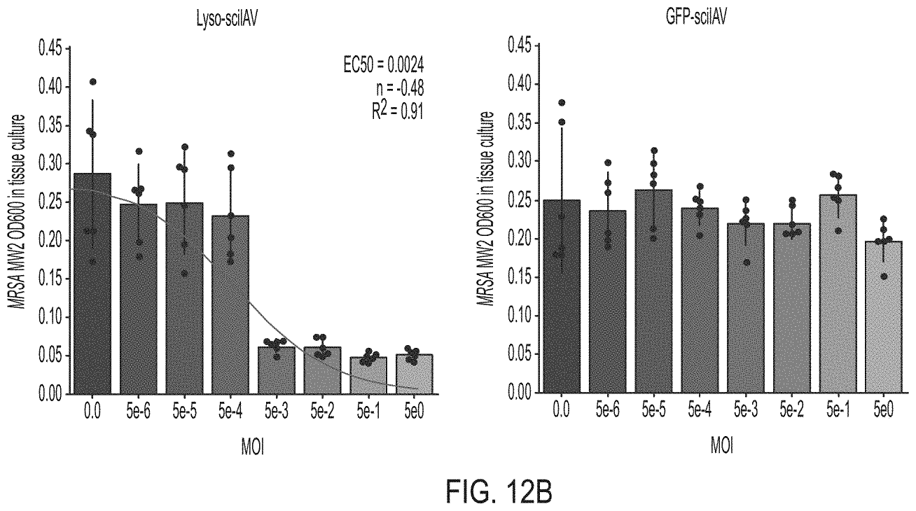

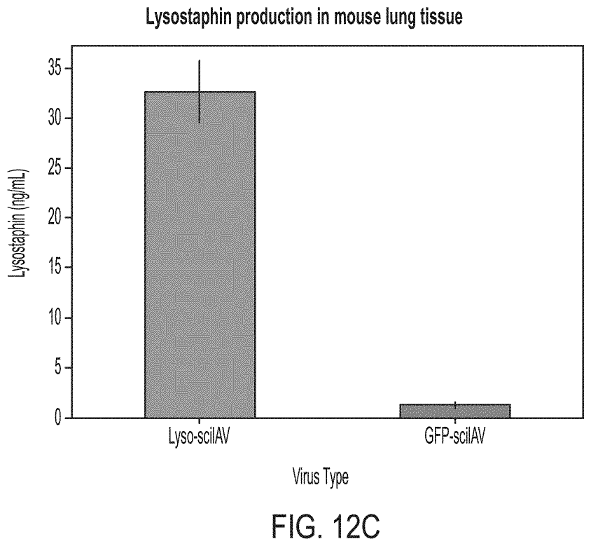

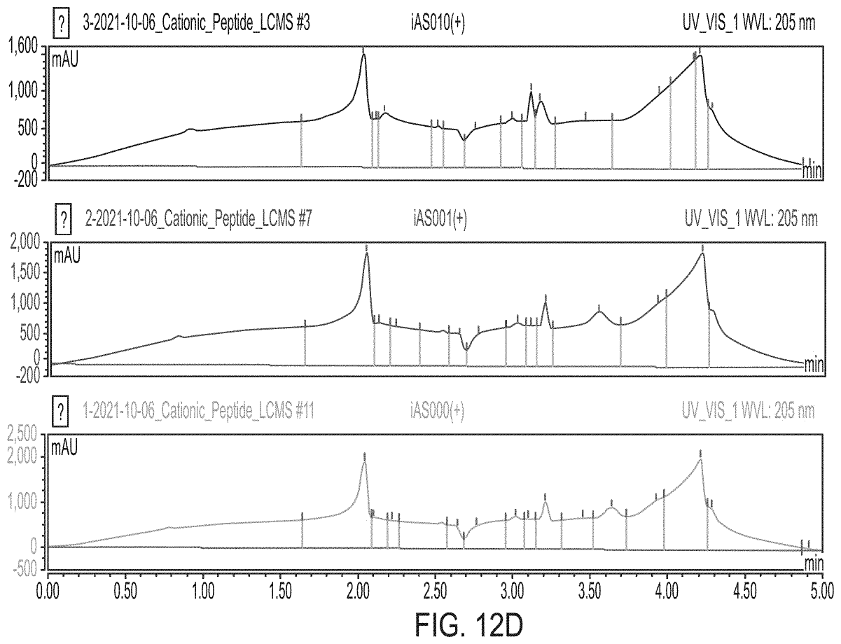

Having characterized key properties of the domesticated virus, we wanted to display our system's ability to take a naturally pathogenic organism and use it for therapeutic application. Though lysostaphin has attracted attention as a method to combat drug resistant strains of Staphylococcus aureus, delivery to infected tissues and bioavailability remain barriers to its application. We sought to take advantage of the transient genetic delivery our rIAV vector system enables to bring lysostaphin directly to the site of infection. We initially characterize the antimicrobial potential of our system through a standard growth inhibition assay. We transduced 10⁶ HEK293t 5 MOI of either lyso-sciIAV, GFPsciIAV, or no virus. Supernatant from lyso-sciIAV transduced S. aureus cell lines created a zone of inhibition when spotted onto LB plates coated with MW2, a high-virulence community-acquired MRSA strain. This zone was comparable in size to that of 1 μg of Kanamycin. GFP-sciIAV, media from untreated cells, clean media, or PBS were unable to induce any zone of inhibition, confirming that active lysostaphin was being produced and secreted in our system (FIGS. 12A-12D).

Low-Dose Protection of Cell Culture from Bacterial Infection.

As an alternative use case, we next sought to demonstrate protective activity of lyso-sciIAV in culture. We transduced HEK293t at 5*10⁻⁶-5*10° MOI of lyso-sciIAV. At 24 hours, we directly contaminated the cell culture with 2 μL of S. aureus RN4220 liquid culture. Addition of virus did not elicit any significant difference on absorbance in the absence of bacterial contamination. In contaminated samples, however, lyso-sciIAV was able to completely inhibit the growth of S. aureus with doses as low as 0.005 MOI. The reported minimal inhibitory concentration for lysostaphin is reported as 0.004 μg/mL, implying that lysostaphin is produced at an estimated 1 pg per infected cell. Similar inhibition was seen is S. aureus MW2 in a separate experiment. To confirm that the lysostaphin cargo, not the sciIAV platform, acts as the antimicrobial agent, we repeated the experiment with GFP-sciIAV. The sciIAV strain carrying GFP cargo exhibited no inhibition of S. aureus. Interestingly, GFP fluorescence was below the detection limit of our plate reader at 0.005 MOI GFP-sciIAV transduction, speaking to the potency of lysostaphin.

In Mice.

A major promise of our transient, non-integrating sciIAV platform is the potential for delivery of antimicrobial peptides to lung tissue as a prophylactic or post-exposure therapy. To ensure that delivery could occur in vivo, we treated C57BL/6 mice with 10⁵ PFU of either lyso-sciIAV or GFP-sciIAV. Using ELISA, we found that lyso-sciIAV infected mice generated 32.6±3.4 ng/mL of lysostaphin, more than 8-fold the minimal inhibitory concentration. The mice exhibited no adverse symptoms from the treatment. Following the success of the lysostaphin producing strain we wanted to repurpose our platform to deliver a broad spectrum antimicrobial peptide. We chose to pursue FK-13, a broad spectrum antimicrobial peptide, derived from the human LL-37 protein. To best take advantage of the short nature of these peptides, we designed our system to encode 5 repeats interspaced with furin cleavage sites, all downstream of the gLuc signal peptide.

Methods

Strains and Chemicals.

IAV strains were stored at −80° C. All bacterial strains were stored at −80° C. in 20% glycerol solution.

Plasmids.

pDZ-PB1, pDZ-PB2, pDZ-PA, pDZ-NP, and pCAGGS-HA were generous gift of the Langlois lab. pDZ-NA, pDZ-NS, pDZ-M were generated by amplifying the pDZ-PB1 backbone and using Gibson assembly to insert ds-DNA blocks encoding the appropriate PR8 segments. HA-packaged effector plasmids were generated using a Gibson reaction.

Cell Culture.

All cells were maintained at 37° C. and 5% CO₂. Gibco DMEM with GlutaMax was supplemented with 10% Fetal Bovine Serum.

Production of Single-Cycle rIAV.

10⁶ HEK293t cells were plated in either a 6 well plate or a 3 cm dish one day before transfection. The cells were transfected with 500 ng of each pDZ plasmid (PB1, PB2, PA, NP, NA, NS, and M), pCAGGS-HA, and the desired HA-packaged effector. 24 hours post transfection, 7.5*10⁵ MDCK-HA cells were overlayed on the transfected HEK293ts with 1 μg/mL of TPCK-treated trypsin. When approximately 70% of the cell had died, 500 μL of supernatant was transferred to a confluent T-75 flask of MDCK-HA cells in OptiMEM media with 1 μg/mL of TPCK-treated trypsin. When approximately 70% of the cells had died, the was collected, centrifuges at 500 rcf for 5 min. The supernatant was separated into 500 μL aliquots and immediately frozen at −80° C.

RNA Collection and RT-PCR.

Immediately before collection RNA lysis buffer was made. Briefly, a 10 mM Tris pH 7.4, 0.25% Igepal CA-630, 150 mM NaCl solution was made in H₂O. Media was aspirated from the cells in a 96 well plate. 200 uL of the lysis buffer was added to the cells and incubated for 20 minutes at room temperature. The LunaScript RT Master Mix Kit was used to generate cDNA using primers oAS344 for cRNA and oAS345 for vRNA (Sup.). oAS344 and oAS345 were then both used to amplify the cDNA.

### Example 3

### rIAV Mediated Delivery of Antibiotic Proteins

Growth inhibition by lysostaphin. Media was collected from cells infected with iAS008, iAS001, or no virus. Media and alternative control treatments were diluted from 10° to 10⁻⁵. An agar plate was coated with Staphylococcus aureus MW2, a virulent kanamycin sensitive strain of MRSA. 5 μL of treatment solutions were spotted. Zones of inhibition were photographed 24 hrs later. 5 μL of iAS008 media has comparable inhibition to 1 μg of Kanamycin. This effect is specific to the lysostaphin encoding portion of the rIAV.

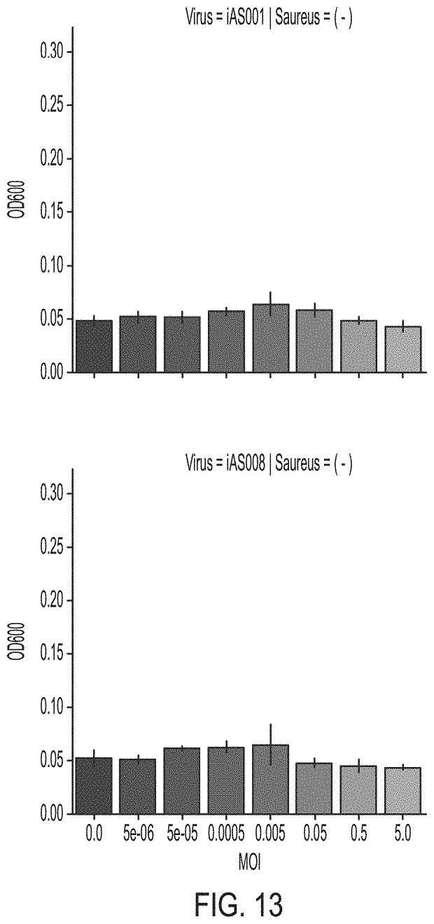

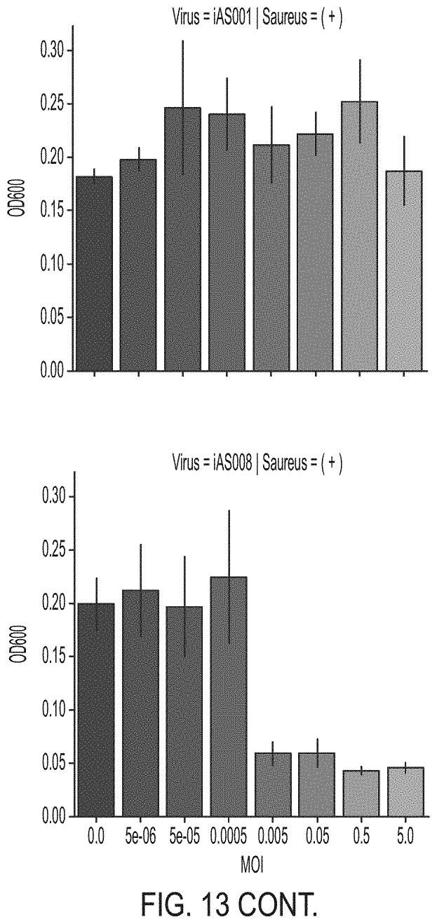

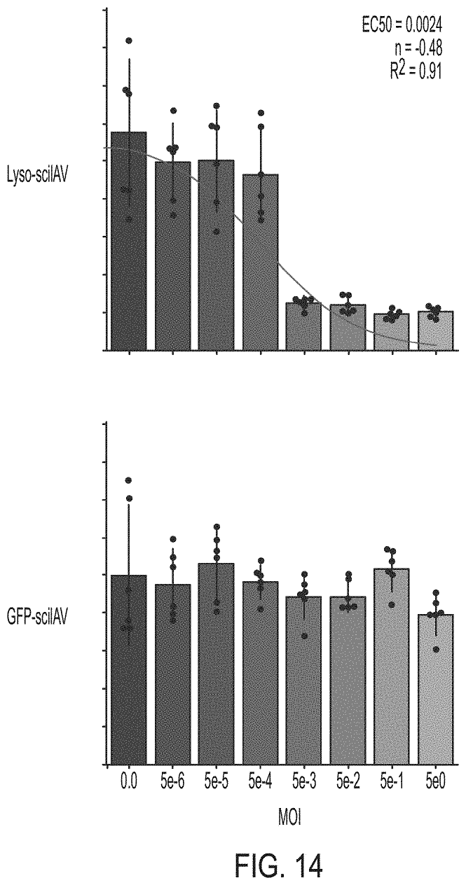

S. aureus inhibition in cell culture. Cells were treated with dilutions of iAS008 or iAS001. Cultures were contaminated with S. aureus. MW2. OD600 was measured 24 hrs later as a proxy for bacterial cell density. Results are provided in FIG. 13. At ≥0.005 MOI of iAS008 growth of S. aureus is fully suppressed. Active lysostaphin is produced in the media. FIG. 14 provides the same data as FIG. 13, but with hill function fit.

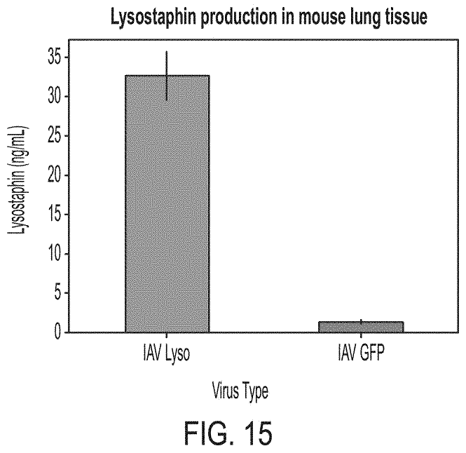

rIAV enables in vivo lysostaphin production. Mice were given iAS008 or iAS001 intranasally. Twenty-four hours later ELISA was used to measure lung concentration of lysostaphin in the lungs of sacrificed animals. Lysostaphin concentration in the lungs is 8-fold the minimal inhibitory concentration. FIG. 15.

rIAV platform produces broad spectrum antibiotic, FK-13 as shown in FIG. 12D.

### Example 4

### rIAV Platform for Versatile Delivery of Effector Protein Encoding RNA

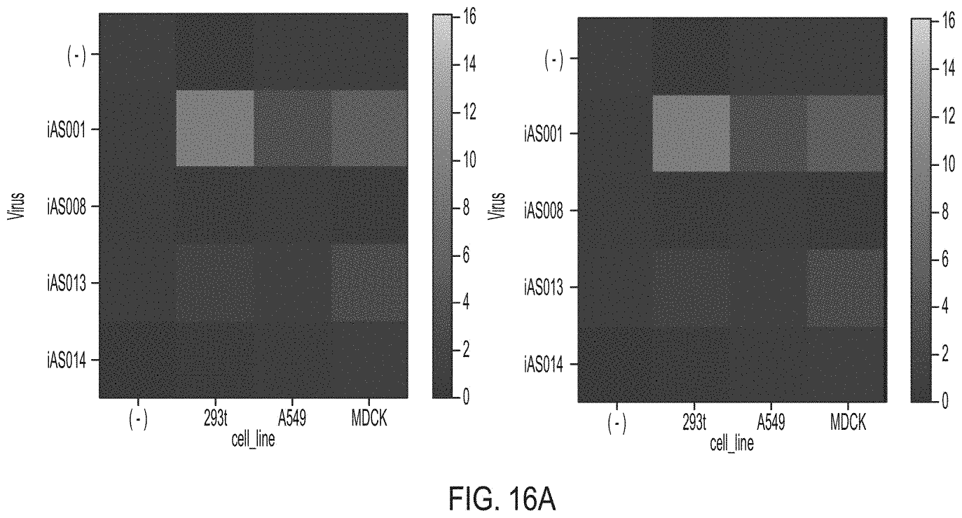

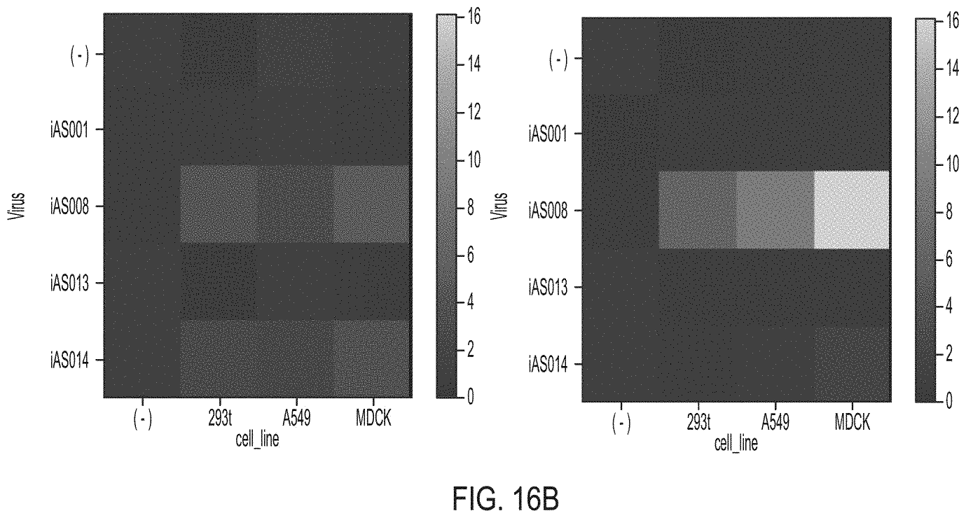

qRT-PCR of iAS001, iAS008 infected cells confirms production of RNA species encoding the desired product in a range of cell types. Three cell lines (and a no-cell control) were transduced with either iAS001 or iAS008 (iAS013 and iAS014 encode GFP and lysostaphin respectively with modified regulatory region). Strand specific cDNA synthesis was used to quantify either negative strand vRNA or a combination of both positive strand species (cRNA+mRNA). All four cell lines generated both strands for the delivered sequence. FIGS. 16A-16B.

### Example 5

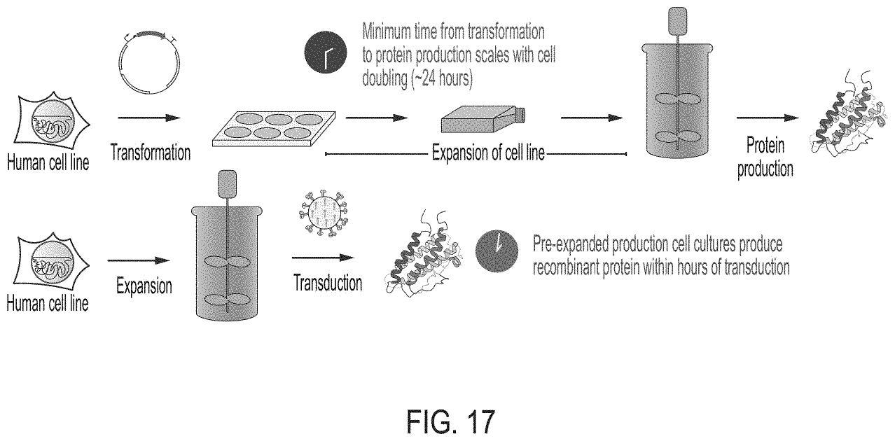

The rIAV platform serves as an alternative pipeline for traditional biomanufacturing. FIG. 17. Top: Traditionally, cell lines must be transformed, selected for production, and grown to scale before protein is collected. Long (˜24 hr) doubling times are exasperated if the cell line has any fitness penalty that comes with protein production. Bottom: Wild-type cell lines can be pre-expanded at normal doubling times ahead of production. Addition of rIAV rapidly reprograms cells for biomanufacturing without expansion delays.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. An isolated replication defective non-integrating segmented virus comprising a genome where at least one of the viral segments comprises a heterologous nucleotide sequence comprising a nucleotide segment that encodes an effector protein.

2. The virus of claim 1, which is an influenza virus, which comprises an HA viral segment.

3. The virus of claim 1, wherein the heterologous nucleotide sequence comprises a secretory signal sequence operably linked to the nucleotide segment.

4. The virus of claim 3, wherein the signal sequence has at least 80% amino acid sequence identity to one of SEQ ID Nos. 11-28.

5. The virus of claim 1, wherein the effector protein is an antimicrobial protein.

6. The virus of claim 5, wherein the antimicrobial protein lyses bacteria.

7. The virus of claim 5, wherein the antimicrobial protein comprises lysostaphin.

8. The virus of claim 5, wherein the antimicrobial protein comprises a sequence having at least 80% amino acid sequence identity to one of SEQ ID Nos. 1-10 or 29-37, or a portion thereof with antimicrobial activity.

9. The virus of claim 1, wherein the effector protein is a SsoPox protein, SARS-CoV-2 spike protein, Anti-SARS-CoV-2-ScFv, ACE2 Receptor Mannan-binding lectin, FK-13, Erythropoietin.

10. The virus of claim 1, wherein the effector protein comprises a sequence having at least 80% amino acid sequence identity to one of SEQ ID Nos. 38-46, or a portion thereof with biological activity.

11. The virus of claim 1, wherein the genome further comprises a heterologous nucleotide segment that encodes a heterologous targeting ligand.

12. The virus of claim 2, wherein the HA viral segment comprises the heterologous nucleotide sequence.

13. The virus of claim 2, wherein an HA coding sequence in the HA viral segment is disrupted so as not to encode a functional HA.

14. A method to prevent, inhibit or treat microbial infection in an animal, comprising administering to the animal an effective amount of a composition comprising the isolated replication defective non-integrating segmented virus of claim 1.

15. The method of claim 14, wherein the composition is delivered locally, systemically, intranasally, or topically.

16. The method of claim 14, wherein the animal is an avian or a mammal.

17. The method of claim 14, wherein the animal has a bacterial infection.

18. The method of claim 17, wherein the mammal has a Staphylococcus, Proteus, Listeria, or Pseudomonas infection.

19. The method of claim 14, wherein the animal has a fungal infection.

20. The method of claim 2, wherein the antimicrobial protein comprises a cutonase-like serine esterase, defensin, indolicidin, protegrain, LL-37, S-type pyocin or tailocin.


SEQUENCE LISTING (51 sequences)

SEQ ID NO: 1 - protein, 263 aa, Artificial Sequence
  Description of Artificial Sequence: Synthetic polypeptide
MGVKVLFALICIAVAEAAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSQSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWQKSTNTLGVLWGTIK

SEQ ID NO: 2 - protein, 246 aa, Artificial Sequence
  Description of Artificial Sequence: Synthetic polypeptide
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 3 - protein, 274 aa, Artificial Sequence
  Description of Artificial Sequence: Synthetic polypeptide
VETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 4 - protein, 668 aa, Artificial Sequence
  Description of Artificial Sequence: Synthetic polypeptide
MVLQQTEPTDGADRKASDGPLTVTAPVPYAAGPTLRNPFPPIADYGFLSDCETTCLISSAGSVEWLCVPRPDSPSVFGAILDRGAGHFRLGPYGVSVPAARRYLPGSLILETTWQTHTGWLIVRDALVMGPWHDIDTRSRTHRRTPMDWDAEHILLRTVRCVSGTVELVMSCEPAFDYHRVSATWEYSGPAYGEAIARASRNPDSHPTLRLTTNLRIGIEGREARARTRLTEGDNVFVALSWSKHPAPQTYEEAADKMWKTSEAWRQWINVGDFPDHPWRAYLQRSALTLKGLTYSPTGALLAAPTTSLPETPQGERNWDYRYSWIRDSTFALWGLYTLGLDREADDFFSFIADVSGANNGERHPLQVMYGVGGERSLVEEELHHLSGYDNSRPVRIGNGAYNQRQHDIWGTMLDSVYLHAKSREQIPDALWPVLKNQVEEAIKHWKEPDRGIWEVRGEPQHFTSSKIMCWVALDRGSKLAELQGEKSYAQQWRAIAEEIKADVLARGVDKRGVLTQRYGDDALDASLLLAVLTRFLPADDPRIRATVLAIADELTEDGLVLRYRVEETDDGLAGEEGTFTICSFWLVSALVEIGEISRAKHLCERLLSFASPLHLYAEEIEPRTGRHLGNFPQAFTHLALINAVVHVIRAEEEADSSGVFVPANAPM

SEQ ID NO: 5 - protein, 92 aa, Artificial Sequence
  Description of Artificial Sequence: Synthetic polypeptide
MKFFVLVAIAFALLACVAQAQPVSDVDPIPEDHVLVHEDAHQEVLQHSRQKRATCDLLSKWNWNHTACAGHCIAKGFKGGYCNDKAVCVCRN

SEQ ID NO: 6 - protein, 13 aa, Artificial Sequence
  Description of Artificial Sequence: Synthetic peptide
ILPWKWPWWPWRR

SEQ ID NO: 7 - protein, 57 aa, Artificial Sequence
  Description of Artificial Sequence: Synthetic polypeptide
MKFFVFALVLALMISMISADSHEKRHHGYRRKFHEKHHSHREFPFYGDYGSNYLYDN

SEQ ID NO: 8 - protein, 149 aa, Artificial Sequence
  Description of Artificial Sequence: Synthetic polypeptide
METQRASLCLGRWSLWLLLLALVVPSASAQALSYREAVLRAVDRLNEQSSEANLYRLLELDQPPKADEDPGTPKPVSFTVKETVCPRPTRQPPELCDFKENGRVKQCVGTVTLDQIKDPLDITCNEVQGVRGGRLCYCRRRFCVCVGRG

SEQ ID NO: 9 - protein, 860 aa, Artificial Sequence
  Description of Artificial Sequence: Synthetic polypeptide
MAQQTHYDGTSGSVVITSGPPASSGGSGGGFGGGGGVSGGFGRTSKRRQKARRRAIEADRQKKEKERAQAEAEAQAEAAQAQAAAAQAQEQARVQARHQQLEGLAQHHAAVRVEVDQRFAARSAQLAPTLEQEVLAARRPPDIHLSERLQLHNITKQKQEVDGLITRKTAELNAKNAVARSFDGHDPLTRTVNDYRARLEQFGEALVHGHQTWENAYNAAHEARLLSTQISALTGKSSALARRHAEQTIVWREREAVWEAQRQYARQREARVRFKQQADEDARVERVRQANTLTVPVSSLTAGGMALTRDGIFVAQQGAAVLEMAVQSAVNTLIDFGRIAAKTGPVFVTAMVYSPTLGDGELSAEQRRRLFQAVGVPAPTLGLTDRQALQSVADAGGSVEVAYRLKSETVPEGTAIIAVATGDAIGADVPVINAVLDPLTGLYSAEIPGSPARHLQFTPDTTQVATASQPGLSVLTPQVEAIAAGVDLRISDCIVCVPDQPPIYFTFNLPPIGSGVVTGVGKPAANDWWKAASQAQGAAIPVQIGDQFRGREFNSLAAFDEALWRTLGEHPTLIAQFDEVNKKRIEQGFAPYAPKSTWVGERREFELRYQERAELGANPFNLDKISITTPQSIQGRLGITPAVLPWPIRPVGVGTWTPLVPPGIEHLGPTTLPVTPSIPAVYPGTPIIPVLPQNETFPAVDEGQIGASIPGYPADMELPSPDVLFLDRRDDPGVAMGVGQSVSGIWLGDAARTEGAPIPEQIADKLRSMEFRNFHGFRRAFWRAVAADAGLSQQFSKSNLDRMRDGSAPFSVLVDQVGGRKAFELHHQVEISKGGDVYGLDNISVMTPKRHINLHKENNQ

SEQ ID NO: 10 - protein, 77 aa, Artificial Sequence
  Description of Artificial Sequence: Synthetic polypeptide
MENKESGEKFSWLEVFKQVVISGFTGFLAGIYGYEQGYSEFITMAFSGLGGALGGHLLDLLWKRLTNSLEKENHSKH

SEQ ID NO: 11 - protein, 25 aa, Homo sapiens
MGVLLTQRTLLSLVLALLFPSMASM

SEQ ID NO: 12 - protein, 16 aa, Vesiculovirus indiana
MKCLLYLAFLFIGVNC

SEQ ID NO: 13 - protein, 21 aa, Mus sp.
METDTLLLWVLLLWVPGSTGD

SEQ ID NO: 14 - skipped

SEQ ID NO: 15 - protein, 19 aa, Homo sapiens
MGWSCIILFLVATATGVHS

SEQ ID NO: 16 - protein, 17 aa, Unknown
  Description of Unknown: BM40 sequence
MRAWIFFLLCLAGRALA

SEQ ID NO: 17 - protein, 21 aa, Unknown
  Description of Unknown: Secrecon sequence
MWWRLWWLLLLLLLLWPMVWA

SEQ ID NO: 18 - protein, 22 aa, Homo sapiens
MDMRVPAQLLGLLLLWLRGARC

SEQ ID NO: 19 - protein, 16 aa, Unknown
  Description of Unknown: CD33 sequence
MPLLLLLPLLWAGALA

SEQ ID NO: 20 - protein, 23 aa, Unknown
  Description of Unknown: tPA sequence
MDAMKRGLCCVLLLCGAVFVSPS

SEQ ID NO: 21 - protein, 18 aa, Homo sapiens
MAFLWLLSCWALLGTTFG

SEQ ID NO: 22 - protein, 15 aa, Homo sapiens
MNLLLILTFVAAAVA

SEQ ID NO: 23 - protein, 20 aa, Homo sapiens
MYRMQLLSCIALSLALVTNS

SEQ ID NO: 24 - protein, 17 aa, Gaussia sp.
MGVKVLFALICIAVAEA

SEQ ID NO: 25 - protein, 16 aa, Homo sapiens
MKWVTFISLLFSSAYS

SEQ ID NO: 26 - protein, 16 aa, Influenza A virus
MKTIIALSYIFCLVLG

SEQ ID NO: 27 - skipped

SEQ ID NO: 28 - protein, 16 aa, Bombyx mori
MKPIFLVLLVVTSAYA

SEQ ID NO: 29 - protein, 186 aa, Artificial Sequence
  Description of Artificial Sequence: Synthetic polypeptide
EPCSDIEVVFARGTSEPAGIGRVGQALTDAIRNQVGGRTVSTYGVNYPATYDFLAAADGANDATNRIATLAEQCPSTRVVLGGYSQGAAVVDMLLGIPPLGNKVGNFGSAPPLPSNLMNNVAAVAVFGNPSAKFGIPVTSRFGGRAIDACSDGDPICSDGRNPFAHTHYESSPFIPQAAGLIAGLV

SEQ ID NO: 30 - protein, 170 aa, Artificial Sequence
  Description of Artificial Sequence: Synthetic polypeptide
MKTQRDGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

SEQ ID NO: 31 - protein, 19 aa, Artificial Sequence
  Description of Artificial Sequence: Synthetic peptide
  MOD_RES (19)..(19): Any amino acid
RGGRLCYCRRRFCVCVGRX

SEQ ID NO: 32 - protein, 13 aa, Artificial Sequence
  Description of Artificial Sequence: Synthetic peptide
ILPWKWPWWPWRR

SEQ ID NO: 33 - protein, 57 aa, Artificial Sequence; identical to SEQ ID NO: 7
  Description of Artificial Sequence: Synthetic polypeptide

SEQ ID NO: 34 - protein, 94 aa, Artificial Sequence
  Description of Artificial Sequence: Synthetic polypeptide
MRTLAILAAILLVALQAQAEPLQARADEVAAAPEQIAADIPEVVVSLAWDESLAPKHPGSRKNMACYCRIPACIAGERRYGTCIYQGRLWAFCC

SEQ ID NO: 35 - protein, 67 aa, Artificial Sequence
  Description of Artificial Sequence: Synthetic polypeptide
MRIHYLLFALLFLFLVPVPGHGGIINTLQKYYCRVRGGRCAVLSCLPKEEQIGKCSTRGRKCCRRKK

SEQ ID NO: 36 - protein, 498 aa, Artificial Sequence
  Description of Artificial Sequence: Synthetic polypeptide
MSNDNEVPGSMVIVAQGPDDQYAYEVPPIDSAAVAGNMFGDLIQREIYLQKNIYYPVRSIFEQGTKEKKEINKKVSDQVDGLLKQITQGKREATRQERVDVMSAVLHKMESDLEGYKKTFTKGPFIDYEKQSSLSIYEAWVKIWEKNSWEERKKYPFQQLVRDELERAVAYYKQDSLSEAVKVLRQELNKQKALKEKEDLSQLERDYRTRKANLEMKVQSELDQAGSALPPLVSPTPEQWLERATRLVTQAIADKKQLQTTNNTLIKNSPTPLEKQKAIYNGELLVDEIASLQARLVKLNAETTRRRTEAERKAAEEQALQDAIKFTADFYKEVTEKFGARTSEMARQLAEGARGKNIRSSAEAIKSFEKHKDALNKKLSLKDRQAIAKAFDSLDKQMMAKSLEKFSKGFGVVGKAIDAASLYQEFKISTETGDWKPFFVKIETLAAGAAASWLVGIAFATATATPIGILGFALVMAVTGAMIDEDLLEKANNLVISI

SEQ ID NO: 37 - protein, 233 aa, Artificial Sequence
  Description of Artificial Sequence: Synthetic polypeptide
MEIQKKLVDPSKYGTKCPYTMKPKYITVHNTYNDAPAENEVSYMISNNNEVSFHIAVDDKKAIQGIPLERNAWACGDGNGSGNRQSISVEICYSKSGGDRYYKAEDNAVDVVRQLMSMYNIPIENVRTHQSWSGKYCPHRMLAEGRWGAFIQKVKNGNVATTSPTKQNIIQSGAFSPYETPDVMGALTSLKMTADFILQSDGLTYFISKPTSDAQLKAMKEYLDRKGWWYEVK

SEQ ID NO: 38 - protein, 214 aa, Artificial Sequence
  Description of Artificial Sequence: Synthetic polypeptide
MWWRLWWLLLLLLLLWPMVWANITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV

SEQ ID NO: 39 - protein, 740 aa, Artificial Sequence
  Description of Artificial Sequence: Synthetic polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 40 - protein, 262 aa, Artificial Sequence
  Description of Artificial Sequence: Synthetic polypeptide
MGVKVLFALICIAVAEADIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKGGGGSGGGGSGGGGSQMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTV

SEQ ID NO: 41 - protein, 248 aa, Artificial Sequence
  Description of Artificial Sequence: Synthetic polypeptide
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 42 - protein, 312 aa, Artificial Sequence
  Description of Artificial Sequence: Synthetic polypeptide
MWWRLWWLLLLLLLLWPMVWANITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 43 - protein, 314 aa, Artificial Sequence
  Description of Artificial Sequence: Synthetic polypeptide
MRIPLVGKDSIESKDIGFTLIHEHLRVFSEAVRQQWPHLYNEDEEFRNAVNEVKRAMQFGVKTIVDPTVMGLGRDIRFMEKVVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDFGTAKPEYKPKLAPRWSITLIFEDTIPFLKRNGVNEEVIATIFKENPKKFFS

SEQ ID NO: 44 - protein, 80 aa, Artificial Sequence
  Description of Artificial Sequence: Synthetic polypeptide
FKRIVQRIKDFLRRKRFKRIVQRIKDFLRRKRFKRIVQRIKDFLRRKRFKRIVQRIKDFLRRKRFKRIVQRIKDFLRRKR

SEQ ID NO: 45 - protein, 193 aa, Artificial Sequence
  Description of Artificial Sequence: Synthetic polypeptide
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 46 - protein, 223 aa, Artificial Sequence
  Description of Artificial Sequence: Synthetic polypeptide
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 47 - DNA, 40 bp, Artificial Sequence
  Description of Artificial Sequence: Synthetic primer
atgcacacctgctacttcacttaatggtgccccacagtac

SEQ ID NO: 48 - DNA, 38 bp, Artificial Sequence
  Description of Artificial Sequence: Synthetic primer
tgcatcacctgcccatatgggcgtgaaggtcctgttcg

SEQ ID NO: 49 - DNA, 52 bp, Artificial Sequence
  Description of Artificial Sequence: Synthetic primer
atgcacacctgctactccatggctagcctatacaaattgtgtctgcaactgc

SEQ ID NO: 50 - DNA, 49 bp, Artificial Sequence
  Description of Artificial Sequence: Synthetic primer
tgcatcacctgcccatgtgactcgagatctactcaactgtcgccagttc

SEQ ID NO: 51 - protein, 24 aa, Homo sapiens
MALWMRLLPLLALLALWGPDPAAA